(19) United States
(12) Patent Application Publication
(10) Pub. No.: US 2003/0228564 A1
(43) Pub. Date: Dec. 11, 2003

(54) Nitric oxide in a pathogen inactivation process

(76) Inventors: Richard Alan Edrich, Denver, CO (US); Laura Goodrich, Lakewood, CO (US); Reinhold Deppisch, Hechingen (DE); Dennis J. Hlavinka, Arvada (CO); Robert Owen Lockerbie, Littleton, CO (US); Werner Beck, Rottenburg (DE)

Correspondence Address: GREENLEE WINNER AND SULLIVAN P C, 5370 MANHATTAN CIRCLE, SUITE 201, BOULDER, CO, 80303, US

(21) Appl. No.: 10/364,661

(22) Filed: Feb. 10, 2003

Related U.S. Application Data
(63) Continuation-in-part of application No. 10/328,717, filed on Dec. 23, 2002.
(63) Continuation-in-part of application No. 10/159,781, filed on May 30, 2002.
(60) Provisional application No. 60/355,393, filed on Feb. 8, 2002.
(60) Provisional application No. 60/373,936, filed on Apr. 19, 2002.
(60) Provisional application No. 60/344,109, filed on Dec. 28, 2001.
(60) Provisional application No. 60/294,866, filed on May 30, 2001.

(51) Int. Cl. A01N 1/02; A61K 31/7076; A61K 31/198; A61K 33/00

(52) U.S. Cl. 435/2; 424/718; 514/47; 514/48; 514/562; 514/565

(57) ABSTRACT

This invention provides methods and compositions for using nitric oxide in a photoradiation pathogen inactivation process for whole blood and blood components to improve pathogen kill and to improve preservation of the quality of the blood components. This invention provides methods for using nitric oxide in combination with oxygen, photosensitizers, quencher and/or glycolysis inhibitor, and compositions comprising blood components decontaminated by these methods. Nitric oxide is provided using nitric oxide gas, or nitric oxide generators such as L-arginine, and/or N-acetyl-cysteine. This invention also provides compositions suitable for photoradiation pathogen inactivation that include fluid comprising a blood component, a photosensitizer, and dissolved nitric oxide. This invention provides decontamination systems useful for performing the methods of this invention and methods for making the decontamination systems. This invention also provides methods for decontaminating fluids and methods for increasing the storage life and quality of photochemically decontaminated platelets.

### CROSS-REFERENCE TO RELATED APPLICATIONS

This application claims priority to U.S. Provisional Application No. 60/355,393, filed Feb. 8, 2002 and to U.S. Provisional Application No. 60/373,936 filed Apr. 19, 2002. This application is also a continuation-in-part of U.S. patent application Ser. No. 10/328,717, filed Dec. 23, 2002, which claims priority to U.S. Provisional Application No. 60/344,109, filed Dec. 28, 2001. This application is also a continuation-in-part of U.S. patent application Ser. No. 10/159,781, filed May 30, 2002, which claims priority to U.S. Provisional Application No. 60/294,866, filed May 30, 2001. This application is a continuation-in-part of U.S. patent application entitled “Inactivation of West Nile Virus and Malaria Using Photosensitizers,” attorney docket number 3-03, filed Feb. 3, 2003, which claims priority to U.S. Provisional Application No. 60/353,162, filed Feb. 1, 2002, and also to U.S. application Ser. No. 09/586,147, filed Jun. 2, 2000 which is a continuation-in-part of U.S. application Ser. No. 09/357,188 filed Jul. 20, 1999 which is a continuation-in-part of U.S. application Ser. No. 09/119,666 filed Jul. 21, 1998. This application is a continuation-in-part of U.S. patent application entitled “Addition of Glycolysis Inhibitor to a Pathogen Reduction and Storage Solution,” attorney docket number B-0112, filed Feb. 3, 2003, that claims priority to U.S. Provisional Application 60/353,319, filed Feb. 1, 2002. This application is a continuation-in-part of U.S. patent application Ser. No. 09/777,727, filed Feb. 5, 2001, which is a continuation-in-part of U.S. patent application Ser. No. 09/420,652, filed Oct. 19, 1999. All of these applications are hereby incorporated by reference to the extent not inconsistent with the disclosure herewith.

### BACKGROUND

Contamination of blood supplies with infectious microorganisms such as malaria, West Nile virus, HIV, hepatitis and other viruses and bacteria presents a serious health hazard for those who must receive transfusions of whole blood or administration of various blood components such as platelets, red cells, blood plasma, Factor VIII, plasminogen, fibronectin, anti-thrombin III, cryoprecipitate, human plasma protein fraction, albumin, immune serum globulin, prothrombin complex, plasma growth hormones, and other components isolated from blood. Blood screening procedures may miss contaminants, and sterilization procedures, which do not damage cellular blood components but effectively inactivate all infectious viruses and other microorganisms have not heretofore been available. In addition, a system that uses the same chemistry to inactivate microorganisms in different fluids, for example separate blood components, is desired for many reasons, including ease of use in a blood bank setting. This type of system has not heretofore been available. It is also desired that the inactivation treatment be easily implemented in a blood bank setting, and produce inactivation in a short period of time.

Whole blood collected from volunteer donors for transfusion recipients is typically separated into its components: red blood cells, platelets, and plasma by apheresis or other known methods. Each of these fractions are individually stored and used to treat a multiplicity of specific conditions and disease states. For example, the red blood cell component is used to treat anemia, the concentrated platelet component is used to control bleeding, and the plasma component is used frequently as a source of Clotting Factor VIII for the treatment of hemophilia.

In the United States, blood storage procedures are subject to regulation by the government. The maximum storage periods for the blood components collected in these systems are specifically prescribed. For example, whole blood components collected in an “open” (i.e., non-sterile) system must, under governmental rules, be transfused within twenty-four hours and in most cases within six to eight hours. By contrast, when whole blood components are collected in a “closed” (i.e., sterile) system the red blood cells can be stored up to forty-two days (depending upon the type of anticoagulant and storage medium used) and plasma may be frozen and stored for even longer periods.

While red cells are stored in the cold, Murphy and Gardner, New Eng. J. Med. 280:1094 (1969), demonstrated that platelets stored as platelet-rich plasma (PRP) at 22° C. possessed a better in vivo half-life than those stored at 4° C. Thus, more acceptable platelet concentrates could be transfused after storage at room temperature. Until recently, the rules allowed for platelet concentrate storage at room temperature for up to seven days (depending upon the type of storage container). However, it was recognized that the incidence of bacterial growth and subsequent transfusion reactions in the recipient increased to unacceptable levels with a seven-day-old platelet concentrate. Platelet concentrates may currently be stored for no more than five days.

Bacteria can easily be introduced to blood components by at least two different means. First, if the donor is experiencing a mild bacteremia, a condition comprising bacteria in the blood, the blood will be contaminated, regardless of the collection or storage method. Adequate donor histories and physicals will decrease but not eliminate this problem. See B. J. Grossman et al., Transfusion 31:500 (1991).

A second, more pervasive source of contamination is the venepuncture. Even when “sterile” methods of skin preparation are employed, it is extremely difficult to sterilize the crypts around the sweat glands and hair follicles. During venepuncture, this contaminated skin is often cut out in a small “core” by a sharp needle. This core can serve to “seed” the blood bag with bacteria that may grow and become a risk to the recipient.

Indeed, many patients requiring platelet transfusions lack host-defense mechanisms for normal clearing and destruction of bacteria because of either chemotherapy or basic hematologic disease. The growth of even seemingly innocuous organisms in stored platelets can, upon transfusion, result in recipient reaction and death. See e.g., B. A. Myhre, JAMA 244:1333 (1980) and J. M. Heal et al., Transfusion 27:2 (1987).

Pathogen eradication technologies (PET) for blood components using visible light and oxygen or using ultraviolet (UV) light and no gas, are described in: U.S. patent application Ser. No. 10/104,766, filed Mar. 21, 2002; U.S. patent application Ser. No. 10/247,262 filed Sep. 18, 2002; U.S. provisional application serial No. 60/368,778, filed Mar. 28, 2002; U.S. patent application Ser. No. 10/159,781, filed May 30, 2002; U.S. patent application Ser. No. 09/982,298, filed Oct. 16, 2001; U.S. patent application Ser. No. 10/328,717, filed Dec. 23, 2002; U.S. patent application Ser. No. 10/065,073, filed Sep. 13, 2002; U.S. patent application Ser. No. 09/962,029, filed Sep. 25, 2001; U.S. provisional application serial No. 60/353,223, filed Feb. 1, 2002; U.S. provisional application serial. No. 60/355,393, filed Feb. 8, 2002; U.S. provisional application serial No. 60/377,697, filed May 3, 2002; U.S. patent application Ser. No. 10/325,402, filed Dec. 20, 2002; U.S. provisional application serial No. 60/353,319, filed Feb. 1, 2002; U.S. provisional application Ser. No. 60/379,328, filed May 8, 2002; U.S. provisional application serial No. 60/375,734, filed Apr. 26, 2002; U.S. provisional application serial No. 60/373,198, filed Apr. 16, 2002; U.S. provisional application serial No. 60/373,936, filed Apr. 19, 2002; U.S. provisional application serial No. 60/378,374, filed May 6, 2002; U.S. provisional application serial No. 60/375,849, filed Apr. 24, 2002; U.S. patent application Ser. No. 09/586,147, filed Jun. 2, 2000; U.S. patent application Ser. No. 09/596,429, filed Jun. 15, 2000; U.S. provisional application serial No. 60/375,670, filed Apr. 26, 2002; PCT patent application serial. No. PCT/US02/21925, filed Jul. 12, 2002; U.S. provisional application serial No. 60/319,488, filed Aug. 23, 2002; U.S. provisional application serial No. 60/319,641, filed Oct. 22, 2002; U.S. patent application Ser. No. 09/119,666, filed Jul. 21, 1998 (U.S. Pat. No. 6,258,577); U.S. patent application Ser. No. 09/357,188, filed Jul. 20, 1999 (U.S. Pat. No. 6,277,337); U.S. patent application Ser. No. 09/420,652, filed Oct. 19, 1999 (U.S. Pat. No. 6,268,120); U.S. patent application Ser. No. 09/777,727, filed Feb. 5, 2001; U.S. patent application Ser. No. 10/256,852, filed Sep. 26, 2002; U.S. patent application Ser. No. 09/725,426, filed Nov. 28, 2000. The present invention includes all aspects of the cited applications and patents that are not inconsistent with the disclosure herewith. The preceding applications are hereby incorporated by reference to the extent not inconsistent with the disclosure herewith

A visible light process using a one hundred percent oxygen atmosphere and 10 J/Km² energy has delivered good viral inactivation of about 5 logs, bacteria clearance of about 2 logs, and acceptable cell quality. A UV process with 6 J/Km² energy has delivered viral inactivation of about 3 logs, bacteria clearance of about 4 logs, and acceptable cell quality.

It has been found that platelets, which have been treated with a photosensitizer and light to inactivate pathogens, which may be present, may show re-activation of pathogens during long-term storage after such a treatment. In addition to platelet aggregation, platelets may show high GMP-140 expression and low ESC (extended shape change) response by day 5 of storage, both of which may be indications of cytoskeletal changes in the platelets. Such changes may be indications of platelet damage due to the storage conditions. It is therefore necessary to improve the quality of stored photoradiated platelets.

There is a need for methods allowing for better pathogen inactivation while maintaining cell quality above acceptable limits and for methods allowing for improved cell quality while maintaining pathogen inactivation. Large quantities of blood and blood products are discarded by blood banks after certain periods of storage due to expiration of the blood and blood products. By improving the cell quality of blood components after pathogen inactivation and during storage, the shelf life blood components may be increased.

Nitric oxide (NO) is a colorless gas at room temperature and pressure. It is soluble in water and is able to pass through membranes. Nitric oxide is a radical species, but it does not possess the type of reactivity normally associated with other radicals (Fukuto, J. (1995) Advances in Pharmacology 34:1-15). The oxygen-dependent decomposition of nitric oxide in aqueous solution leads to a variety of nitrogen oxide species. In biological fluids, NO exhibits a very short half-life due to a rapid inactivation by reactive oxygen species (ROS). Photoexcitation of membrane-bound agent molecules leads to the formation of ROS such as singlet oxygen. Oxygen free radicals and ROS include superoxide, hydroxy radical, lipid peroxy radical, singlet oxygen, hydrogen peroxide, and hypochlorous acid. One of the most biologically significant aspects of nitric oxide chemistry is its ability to bind and/or react with metals and metal-containing proteins. Examples of metal-binding proteins are the heme-containing enzyme guanylate cyclase and hemoglobin. Nitric oxide is recognized as a noxious air pollutant and toxic cigarette smoke constituent. Many human illnesses have been associated with insufficient synthesis of endogenous nitric oxide (Hanson S. R. et al., (1995) Advances in Pharmacology 34:383).

Recent research has shown that nitric oxide is a powerful biologic mediator acting on many levels (Torreilles, J. (October 2001) Frontiers in Bioscience 6:d1161-1172). Nitric oxide has been shown to be a key messenger molecule for vasodilatation and neurotransmission and to play important roles in cell respiration (Brown, G. C. (May 1999) Biophys Acta 1411(2-3):351-369), immune function, and platelet activation (Sly M. K. et al., (1995) ASAIO J 41:M394-398). It is thought that nitric oxide works in conjunction with natural host defense mechanisms such as macrophages to effect bacterial kill. See Keefer L. K. (August 1998) Chemtech 28(8):30-35 and Wang G. R. et al., (1998) PNAS 95-9:4888-4893. Oxygen may be required for pathogen kill in visible light pathogen inactivation processes using riboflavin and its analogs. Reports in the literature (Zumft, W. G. (1993) “The Biological Role of Nitric Oxide” Arch. Microbiol 160:253-264) indicate that reactive oxygen species (e.g., superoxide) combine with nitric oxide to form a powerful oxidant, peroxynitrite (ONOO—) which can damage DNA (Pacelli, R. et al. (1995) “Nitric Oxide Potentiates Hydrogen Peroxide-Induced Killing of E. coli” J. of Exp. Med. 182:1469-1479 and Hickman-Davis J. M. et al., (2001) Am J Physiol Lung Cell Mol Physiol 281:L517-L523).

Nitric oxide gas has been utilized to kill bacteria in shoes (http://news.bbc.co.uk/2/hi/science/nature/967499.stm, Oct. 11, 2000). Nitric oxide gas had been used as a fumigant to extend the post-harvest life of horticultural produce (http://www.biu.ac.il/birnd/A2HARVES.html, Aug. 1, 2001). Treatment of E. coli with hydrogen peroxide and nitric oxide potentiates hydrogen peroxide killing (Pacelli R. et al., (1995) J. of Exp. Med. 182:1469-1479).

Nitric oxide gas at concentrations of 500 ppm and 1000 ppm have been used in sweep gas of extracorporeal blood oxygenators to improve platelet activation measures (Sly M. K. et al., (1995) ASAIO J 41 :M394-M398; Sly M. K. et al., (1996) ASAIO J 42:M494-M499; http://www.mc3corp.com/nitric_oxide.html) 100 ppm nitric oxide is also effective (Li J. et al., (1999) J. Biomater. Sci. Polymer Edn. 10:(2):235-246). Nitric oxide gas is useful clinically, by inhalation (Kannan, M. et al., (1998) Indian J. Pediatr. 65:333-345 and Frostell C. G. and Zapol W. M. (1995) Advances in Pharmacology 34:439).

Nitric oxide is thought to mediate its effect through cGMP (Sly, M. K. et al., (1997) Shock 8(2):115-118) and cytochrome c oxidase (Brown G. C. (2001) Biochim. et Biophys. Acta 1504:46-57). It is also thought that nitric oxide can impair the bacterial chaperone system, thereby increasing bacterial susceptibility to attack by the host (Kroncke, K. D. (2001) Nitric Oxide: Biology and Chemistry 5(4):289-295). Mitochondrial respiration is inhibited by nitric oxide gas provided by nitric oxide-saturated water (Borutaite V. and Brown G. C. (1996) Biochem J. 315:295-299 and Brown G. C. et al., (1994) FEBS Lett. 356:295-298).

Nitric oxide delivery is useful in wound dressings, containers, sutures, plugs, valves, implants, blood conduits, and other medical devices (Keefer, L. K. (1998) Chemtech 28(8):30-35). Nitric oxide-releasing materials are useful for coating implants (http://www.sciencedaily.com/releases/2001/09/010911073242.htm).

U.S. Pat. No. 6,087,087 describes methods and compositions for treating diluents comprising hemoglobin-containing erythrocytes to increase the oxygen delivery capacity of hemoglobin wherein nitric oxide is bound to hemoglobin. Methods include steps of deoxygenating the erythrocytes. Nitric oxide-related compounds have been described as useful for treating infections and for killing pathologic microbes, helminths, and pathologically proliferating cells (U.S. Pat. Nos. 6,057,367 and 6,180,824). The nitric oxide compounds described in these patents do not include nitric oxide itself.

Prior to this invention, the impact of nitric oxide on photoradiation pathogen inactivation processes and the impact of photoradiation pathogen inactivation processes on the activities of nitric oxide were unknown and could not have been predicted. At high concentrations, nitric oxide was known to be detrimental to cells that require protection and whose qualities require preservation. Even at fairly low concentrations, nitric oxide was known to be toxic to cells (Raghuram, N. et al. (1999) Biochem. and Biophys. Res. Commun. 262:685-691). It was not known how or whether the beneficial effects of nitric oxide could be balanced with its toxic effects. It was thought likely that nitric oxide would have different effects when combined with other components. For example, there is a complex interplay between nitric oxide and oxygen in biological systems (Shiva S. et al. (2001) PNAS 98(13):7212-7217 and Liu X. et al., (1998) PNAS 95:2175-2179). Pathogen photoinactivation processes may require oxygen, which was thought likely to impact the activity of nitric oxide. Light was known to affect chemical and enzymatic reactions involving nitric oxide (http://66.192.44.36/˜xpediti/wills/T/treager1.html). Photoradiation was thought likely to affect the ability of other molecules in a fluid comprising a blood components to be decontaminated by a photoradiation pathogen inactivation process, such as photoactivators or molecules endogenous to blood components, to interact with nitric oxide, and become nitric oxide scavengers, for example. It was known that light could reverse the nitric oxide inhibition of mitochondrial respiration (Brown, G. C. (1999) Biochim. et Biophys. Acta 1411:351-369); hence it was considered likely that nitric oxide in photoradiation pathogen inactivation processes would damage cells and proteins of blood components and possibly lose much or all of its antibacterial qualities as well. Also, problems have been encountered using nitric oxide gas for nitric oxide delivery to biological systems (Pacelli R. et al., (1995) J. of Exp. Med. 182:1469-1479 and U.S. Pat. No. 5,797,887).

Physiological platelet antagonists as well as various pharmacological vasodilators inhibit platelet function by activation of adenyl and guanyly cyclases and increasing intracellular cyclic AMP (cAMP) and cyclic GMP (cGMP) levels, respectively. Elevation of platelet cyclic nucleotides interferes with known platelet activation signaling pathways, and effectively blocks platelet activation. Platelet cAMP and/or cGMP levels may be affected by adding nitric oxide, N-acetyl-cysteine, or L-arginine, directly or indirectly (Schwarz, et al. (2001) Biochem. Pharmacol. 62:1153-1161).

N-acetyl-cysteine is a thiol compound that may inhibit platelet aggregation, and L-arginine has been shown to promote endogenous synthesis of nitric oxide (Anfossi, et al. (2001) Eur. J. of Clin. Invest. 31:452-461). The effect of N-acetyl-cysteine on biological systems has been attributed to an enhancement of bioconversion of nitrates to nitric oxide. N-acetyl-cysteine may enhance the platelet anti-aggregating effect of L-arginine. In vitro studies appear to show that NO interacts with N-acetyl-cysteine to form a NO donor, S-nitroso-N-acetyl-cysteine, which may act as a cGMP activator and inhibit platelet activation.

The anti-aggregatory action of L-arginine may act by stimulating GMP cyclase (Radomski, et al. (1990) PNAS 87:5193-5197. Oral administration of L-arginine has been shown to reduce platelet aggregation (Anfossi, G. et al. (1999) Thrombosis Res. 91:307-316).

Vascular endothelium has been shown to synthesize NO from the terminal guanidino nitrogen atom(s) of L-arginine (Palmer, et al. (1988) Nature 333:664-666).

U.S. Pat. No. 5,552,267 describes solutions for prolonged organ preservation. U.S. Pat. No. 6,365,338 describes compositions and methods of using organ-preserving solutions containing trehalose, anti-oxidant cations, and an energy source. The described solutions may also contain L-arginine.

Methods and compositions for prolonging preservation of blood platelets using inhibitors of secondary messenger effects are described in U.S. Pat. Nos. 5,622,867, 5,919,614, and 6,221,669. The effects of L-arginine and sodium nitroprusside (SNP) are discussed.

U.S. Pat. No. 5,767,160 describes methods and compositions for stimulating nitric oxide synthesis. Compositions include L-arginine and an agonist of nitric oxide synthase.

Therapeutic micronutrient compositions containing N-acetyl-cysteine, L-arginine, and riboflavin for severe trauma, burns, and critical illness are described in U.S. Pat. No. 6,391,332.

PCT Publication WO 02/12207 describes compositions and methods for preventing platelet aggregation using cyclic oxyguanidine protease inhibitors.

PCT Publication WO 98/08482 and U.S. Pat. No. 5,797,887 describe methods and compositions for inhibiting platelet aggregation using foreign bodies, that come in contact with blood, and that are coated or synthesized with nitrosyl-containing organometallic compounds that release nitric oxide.

The use of photosensitizers, compounds which absorb light of a defined wavelength and transfer the absorbed energy to an energy acceptor, has been proposed for blood component sterilization. For example, European Patent Application No. 196,515, published Oct. 8, 1986, suggests the use of non-endogenous photosensitizers such as porphyrins, psoralens, acridine, toluidines, flavine (acriflavine hydrochloride), phenothiazine derivatives, and dyes such as neutral red and methylene blue, as blood additives. Protoporphyrin, which occurs naturally within the body, can be metabolized to form a photosensitizer; however, its usefulness is limited in that it degrades desired biological activities of proteins. Chlorpromazine, is also exemplified as one such photosensitizer; however its usefulness is limited by the fact that it should be removed from any fluid administered to a patient after the decontamination procedure because it has a sedative effect.

Goodrich, R. P., et al. (1997), “The Design and Development of Selective, Photoactivated Drugs for Sterilization of Blood Products,” Drugs of the Future 22:159-171 provides a review of some photosensitizers including psoralens, and some of the issues of importance in choosing photosensitizers for decontamination of blood products. The use of texaphyrins for DNA photocleavage is described in U.S. Pat. Nos. 5,607,924 issued Mar. 4, 1997 and 5,714,328 issued Feb. 3, 1998 to Magda et al. The use of sapphyrins for viral deactivation is described in U.S. Pat. No. 5,041,078 issued Aug. 20, 1991 to Matthews, et al. Inactivation of extracellular enveloped viruses in blood and blood components by Phenthiazin-5-ium dyes plus light is described in U.S. Pat. No. 5,545,516 issued Aug. 13, 1996 to Wagner. The use of porphyrins, hematoporphyrins, and merocyanine dyes as photosensitizing agents for eradicating infectious contaminants such as viruses and protozoa from body tissues such as body fluids is disclosed in U.S. Pat. No. 4,915,683 issued Apr. 10, 1990 and related U.S. Pat. No. 5,304,113 issued Apr. 19, 1994 to Sieber et al. The mechanism of action of such photosensitizers is described as involving preferential binding to domains in lipid bilayers, e.g. on enveloped viruses and some virus-infected cells. Photoexcitation of membrane-bound agent molecules leads to the formation of reactive oxygen species such as singlet oxygen. U.S. Pat. No. 4,727,027 issued Feb. 23, 1988 to Wiesehahn, G. P., et al. discloses the use of furocoumarins including psoralen and derivatives for decontamination of blood and blood products, but teaches that steps must be taken to reduce the availability of dissolved oxygen and other reactive species in order to inhibit denaturation of biologically active proteins.

Photoinactivation of viral and bacterial blood contaminants using halogenated coumarins is described in U.S. Pat. No. 5,516,629 issued May 14, 1996 to Park, and related U.S. Pat. No. 6,251,644 to Sowemimo-Coker et al. issued Jun. 26, 2001. U.S. Pat. No. 5,587,490 issued Dec. 24, 1996 to Goodrich Jr., R. P., et al. and U.S. Pat. No. 5,418,130 to Platz, et al. disclose the use of substituted psoralens for inactivation of viral and bacterial blood contaminants. The latter patent also teaches the necessity of controlling free radical damage to other blood components. U.S. Pat. No. 5,654,443 issued Aug. 5, 1997 to Wollowitz et al. teaches new psoralen compositions used for photodecontamination of blood. U.S. Pat. No. 5,709,991 issued Jan. 20, 1998 to Lin et al. teaches the use of psoralen for photodecontamination of platelet preparations and removal of psoralen afterward. U.S. Pat. No. 5,360,734 issued Nov. 1, 1994 to Chapman et al. addresses the problem of prevention of damage to other blood components. U.S. Pat. No. 5,120,649 issued Jun. 9, 1992, related U.S. Pat. No. 5,232,844 issued Aug. 3, 1993 to Horowitz, et al., related U.S. Pat. No. 5,658,722 issued Aug. 19, 1997 to Margolis-Nunno et al., related U.S. Pat. No. 5,858,643 issued Jan. 12, 1999 to Ben Hur et al., related U.S. Pat. No. 5,981,163 issued Nov. 9, 1999, to Horowitz et al. related U.S. Pat. No. 6,077,659 issued Jun. 20, 2000, to Ben Hur et al. related U.S. Pat. No. 6,214,534 issued Apr. 10, 2001, to Horowitz et al., and related U.S. Pat. No. 6,294,361 issued Sep. 25, 2001 to Horowitz et al. also disclose the need for the use of “quenchers” in combination with photosensitizers which attack lipid membranes. U.S. Pat. Nos. 5,232,844 and 6,294,361 state that the process may be carried out in the presence of an oxidizer, which can be oxygen, and that the concentration of oxygen can be the endogenous quantity, or can be modified by placement of the material being treated in an atmosphere designed to lower or raise oxygen concentration. However, the examples of these patents teach benefits of lowering oxygen content, and of using normal aeration combined with quencher (compared with using a nitrogen atmosphere), thereby effectively teaching against using an increased oxygen concentration. U.S. Pat. No. 5,981,163 teaches benefits of deoxygenization. U.S. Pat. Nos. 6,077,659 and 5,858,643 disclose using vitamin E or derivatives thereof to prevent potassium ion leakage from red blood cells after irradiation with porphyrin-like photosensitizers. U.S. Pat. No. 4,386,069 issued May 31, 1983 to Estep discloses an additive solution to enhance preservation of normal red cell morphology during storage comprising a fatty ester which includes at least two ester linkages comprising fatty hydrocarbon groups of about four to twelve carbon atoms each.

Photosensitizers that attack nucleic acids are known to the art. U.S. Pat. No. 5,342,752 issued Aug. 30, 1994 to Platz et al. discloses the use of compounds based on acridine dyes to reduce parasitic contamination in blood matter comprising red blood cells, platelets, and blood plasma protein fractions. These materials, although of fairly low toxicity, do have some toxicity e.g. to red blood cells. U.S. Pat. No. 5,798,238 to Goodrich, Jr., et al., discloses the use of quinolone and quinolone compounds for inactivation of viral and bacterial contaminants.

Binding of DNA with photoactive agents has been exploited in processes to reduce lymphocytic populations in blood as taught in U.S. Pat. No. 4,612,007 issued Sep. 16, 1986 and related U.S. Pat. No. 4,683,889 issued Aug. 4, 1987 to Edelson.

Riboflavin (7,8-dimethyl-10-ribityl isoalloxazine) has been reported to attack nucleic acids. Photoalteration of nucleic acid in the presence of riboflavin is discussed in Tsugita, A, et al. (1965), “Photosensitized inactivation of ribonucleic acids in the presence of riboflavin,” Biochimica et Biophysica Acta 103:360-363; and Speck, W. T. et al. (1976), “Further Observations on the Photooxidation of DNA in the Presence of Riboflavin,” Biochimica et Biophysica Acta 435:39-44. Binding of lumiflavin (7,8,10-trimethylisoalloxazine) to DNA is discussed in Kuratomi, K., et al. (1977), “Studies on the Interactions between DNA and Flavins,” Biochimica et Biophysica Acta 476:207-217. Hoffmann, M. E., et al. (1979), “DNA Strand Breaks in Mammalian Cells Exposed to Light in the Presence of Riboflavin and Tryptophan,” Photochemistry and Photobiology 29:299-303 describes the use of riboflavin and tryptophan to induce breaks in DNA of mammalian cells after exposure to visible fluorescent light or near-ultraviolet light. The article states that these effects did not occur if either riboflavin or tryptophan was omitted from the medium. DNA strand breaks upon exposure to proflavine and light are reported in Piette, J. et al. (1979), “Production of Breaks in Single- and Double-Stranded Forms of Bacteriophage ΦX74 DNA by Proflavine and Light Treatment,” Photochemistry and Photobiology 30:369-378, and alteration of guanine residues during proflavine-mediated photosensitization of DNA is discussed in Piette, J., et al. (1981), “Alteration of Guanine Residues during Proflavine Mediated Photosensitization of DNA,” Photochemistry and Photobiology 33:325-333.

J. Cadet, et al. (1983), “Mechanisms and Products of Photosensitized Degradation of Nucleic Acids and Related Model Compounds,” Israel J. Chem. 23:420-429, discusses the mechanism of action by production of singlet oxygen of rose bengal, methylene blue, thionine and other dyes, compared with mechanisms not involving production of singlet oxygen by which nucleic acid attack by flavin or pteron derivatives proceeds. Riboflavin is exemplified in this disclosure as having the ability to degrade nucleic acids. Korycka-Dahl, M., et al. (1980), “Photodegradation of DNA with Fluorescent Light in the Presence of Riboflavin, and Photoprotection by Flavin Triplet-State Quenchers,” Biochimica et Biophysica Acta 610:229-234 also teaches that active oxygen species are not directly involved in DNA scission by riboflavin. Peak, J. G., et al. (1984), “DNA Breakage Caused by 334-nm Ultraviolet Light is Enhanced by Naturally Occurring Nucleic Acid Components and Nucleotide Coenzymes,” Photochemistry and Photobiology 39:713-716 further explores the mechanism of action of riboflavin and other photosensitizers. However, no suggestion is made that such photosensitizers be used for decontamination of medical fluids. Korycka-Dahl, M. and Richardson, T. (1980), “Photodegradation of DNA with Fluorescent Light in the Presence of Riboflavin, and Photoprotection by Flavin Triplet-State Quenchers,” Biochimica et Biophysica Acta 610:229-234, discusses the formation of superoxide anions generated upon illumination of nucleic acid in solution with riboflavin. Certain quenchers protected DNA from photodegradation, but alpha-tocopherol did not have a protective effect. The article concluded that active oxygen species are not involved in DNA photodegradation using riboflavin.

Sterilization procedures which do not damage cellular blood components but effectively inactivate infectious viruses and other microorganisms and contaminants are disclosed in U.S. Pat. Nos. 6,258,577, 6,277,337, 6,268,120 and PCT publications WO 01/28599, WO 00/04930, WO 02/26270, WO 02/43485, WO 02/32469, WO 01/96340, WO 01/94349, WO 01/28599, WO 01/23413, and U.S. patent application Ser. Nos. 09/725,426, 09/586,147, 09/777,727, 09/677,375, 09/596,429, 10/247,262, and 10/159,781.

Apparatuses for decontamination of blood have been described in U.S. Pat. No. 5,290,221 issued Mar. 1, 1994 to Wolfe, Jr., et al. and U.S. Pat. No. 5,536,238 issued Jul. 16, 1996 to Bischof. U.S. Pat. No. 5,290,221 discloses the irradiation of fluid in a relatively narrow, arcuate gap. U.S. Pat. No. 5,536,238 discloses devices utilizing optical fibers extending into a filtration medium. Both patents recommend as photosensitizers benzoporphyrin derivatives which have an affinity for cell walls.

Blood separation devices are disclosed, e.g. in PCT publication WO 99/11305 and WO 01/66172.

A few patents discuss the use of additives to quench side reactions. U.S. Pat. No. 6,077,659 (Jun. 20, 2000) to Ben-Hur et al. discusses the use of vitamin E and a phthalocyanine porphyrin-like photosensitizer to inactivate viruses. U.S. Pat. No. 6,270,952 (Aug. 7, 2001) to Cook et al. discusses the use of glutathione with a quinacrine compound to inactivate pathogens.

The breakdown of glucose to provide energy to cells is an important mechanism in cellular metabolism. This mechanism, known as glycolysis, produces ATP (adenosine triphosphate) in the absence of oxygen. The production of ATP is essential for cellular energy metabolism. In the process of glycolysis, a glucose molecule with six carbon atoms is converted into two molecules of pyruvate, each with three carbon atoms. This conversion involves a sequence of nine enzymatic steps that create phosphate-containing intermediates. The cell hydrolyzes two molecules of ATP to drive the early steps, but produces four molecules of ATP in the later steps.

For most animal cells, glycolysis is merely the first stage in the breakdown of sugar into cellular energy, since the pyruvic acid that is formed at the last step quickly enters the cell's mitochondria to be completely oxidized to CO and H₂O in the citric acid cycle. In the case of organisms which are anaerobic (those that do not use molecular oxygen) and for tissues like skeletal muscle that can function under anaerobic conditions, glycolysis can become a major source of the cell's ATP. This also occurs if the mitochondria of the cell are damaged in some way, thereby preventing the cell from entering the citric acid cycle.

Since ATP is essential to continued cell function, when aerobic metabolism is slowed or prevented by lack of oxygen, anaerobic pathways for producing ATP are stimulated and become critical for maintaining cell viability. Here, instead of being degraded in the mitochondria, the pyruvate molecules stay in the cytosol and can be converted into ethanol and CO₂ (as in yeast) or into lactate (as in muscle).

Lactate accumulation in cells causes an increased concentration of hydrogen ions and a decrease in pH. If cells undergoing glycolysis are being stored, such a drop in pH might indicate and contribute to a decrease in cell quality during cell storage.

Factors which might cause cells to enter glycolysis and thereby accumulate lactic acid or lactate may be events which occur internally in a body such as strokes or infarctions, or may be caused by external events such as treatment of the cells after removal from a body. One example of an external treatment, which might cause cells to accumulate lactate, is a procedure to inactivate any pathogens which might be contained in cells to be transfused into a recipient. Currently-used methods to sterilize pathogenic contaminants which may be present in blood or blood components may cause damage to the mitochondria of the cells being treated. If this occurs, the cells can only make ATP through the glycolysis pathway, causing a buildup of lactic acid in the cell, and a subsequent drop in pH during storage.

Glycolysis can be inhibited by glycolysis inhibitors. 2-deoxy-D-glucose is a metabolic inhibitor which slows the rate of glycolysis by inhibiting enzymatic processes within the glycolytic chain. 2-deoxy-D-glucose competes for glucose binding sites within a cell, therefore slowing the rate of glucose utilization and slowing the rate of lactic acid production. Glycolysis inhibitors include 2-deoxy-D-glucose.

There is a need in the art for methods to reduce collateral damage to blood components after treatment with a photosensitizer and light.

All publications and patent applications referred to herein are hereby incorporated by reference to the extent not inconsistent herewith.

### SUMMARY

This invention provides methods and compositions for using nitric oxide, before, during and/or after a photoradiation pathogen inactivation process for blood components to improve pathogen kill and also to improve preservation of the quality of the blood components.

Nitric oxide is a radical species which can be a stable gas in the absence of other species with which it can react. Nitric oxide gas by itself is stable, and it is stable when mixed with nitrogen. In solution, nitric oxide quickly reacts with other species such as oxygen and metal-containing molecules.

This invention provides methods for using nitric oxide in combination with oxygen, photosensitizer, quencher and/or glycolysis inhibitor. This invention provides compositions comprising blood components decontaminated by the methods of this invention. This invention provides compositions suitable for photoradiation pathogen inactivation that include fluid comprising a blood component, a photosensitizer, and dissolved nitric oxide. This invention provides decontamination systems useful for performing the methods of this invention and methods for making the decontamination systems. This invention provides methods for decontaminating fluids and methods for increasing the storage life and quality of photochemically decontaminated platelets.

This invention provides a method for treating a fluid comprising a blood component to inactivate pathogens which may be present therein, comprising the steps of:

(a) adding an inactivation-effective, substantially non-toxic amount of a photosensitizer to said fluid;

(b) adding nitric oxide to said fluid to increase dissolved nitric oxide content of said fluid in an amount sufficient to improve a quality of said blood component; and

(c) exposing said fluid to photoradiation of sufficient energy to activate the photosensitizer, for a sufficient time to substantially inactivate said pathogens.

In an embodiment of this invention, this method also includes one or more steps of adding oxygen, quencher, and/or glycolysis inhibitor. Adding nitric oxide may be performed by adding nitric oxide gas, or nitric oxide generator, including nitric oxide donor. Components that improve cell quality: oxygen, quencher, glycolysis inhibitor, and nitric oxide, may be added before and/or after photoradiation. Nitric oxide generators useful in the practice of this invention include L-arginine and N-acetyl-cysteine. Platelets and other blood components, as well as whole blood, may be decontaminated using the methods and systems of this invention. Pathogens which can be inactivated by the methods of this invention include viruses, bacteria, bacteriophages, fungi, blood-transmitted parasites, and protozoa.

### BRIEF DESCRIPTION OF THE DRAWINGS

FIG. 1 is a graph showing the effect of nitric oxide on GMP-140 expression (% activation) by platelets as a function of storage time (days) using data from Examples 3 and 4.

FIG. 2 is a graph showing the effect of nitric oxide on HSR (% reversal) of platelets as a function of storage time (days).

FIG. 3 is a graph showing the effect of nitric oxide on lactate production (lactate concentration mM/1000 cells) by platelets as a function of storage time (days).

FIG. 4 is a graph showing the effect of nitric oxide on glucose consumption (glucose concentration remaining in fluid mM/1000 cells) by platelets as a function of storage time (days).

FIG. 5 is a graph showing the effect of nitric oxide on BVDV inactivation (log/ml) as a function of energy (J/cm²).

FIG. 6 is a graph showing the effect of nitric oxide on GMP-140 expression (% activation) by platelets as a function of storage time (days).

FIG. 7 is a graph showing the effect of nitric oxide on HSR (% reversal) of platelets as a function of storage time (days).

FIG. 8 is a graph showing the effect of nitric oxide on lactate production (lactate concentration mM/1000 cells) by platelets as a function of storage time (days).

FIG. 9 is a graph showing the effect of nitric oxide on pH of the stored fluid as a function of storage time (days).

FIG. 10 is a graph showing the effect of nitric oxide on platelet swirl (0-3 units) of blood components as a function of storage time (days).

FIG. 11 is a graph showing the effect of nitric oxide on GMP-140 expression (% activation) by platelets as a function of storage time (days).

FIG. 12 is a graph showing the effect of nitric oxide on hypotonic shock response (HSR, % reversal) of platelets as a function of storage time (days).

FIG. 13 is a graph showing the effect of nitric oxide on lactate production (lactate concentration mM/1000 cells) by platelets as a function of storage time (days).

FIG. 14 is a graph showing the effect of nitric oxide on glucose consumption (glucose concentration mM/1000 cells) by platelets as a function of storage time (days).

FIG. 15 is a graph showing the effect of nitric oxide on pH of the stored fluid as a function of storage time (days).

FIG. 16 is a graph showing the effect of nitric oxide on platelet swirl (0-3 units) of platelets as a function of storage time (days).

FIG. 17 is a graph showing the effect of nitric oxide and/or glycolysis inhibitor on GMP-140 expression by platelets as a function of time (days).

FIG. 18 is a graph showing the effect of nitric oxide and/or glycolysis inhibitor on HSR (% reversal) by platelets as a function of storage time (days).

FIG. 19 is a graph showing the effect of nitric oxide and/or glycolysis inhibitor on lactate production (lactate concentration, mM/1000 cells) by platelets as a function of storage time (days).

FIG. 20 is a graph showing the effect of nitric oxide and/or glycolysis inhibitors on glucose consumption (glucose concentration remaining in fluid, mM/1000 cells) by platelets as a function of storage time (days).

FIG. 21 is a graph showing the effect of nitric oxide and/or glycolysis inhibitors on pH of the stored fluid as a function of storage time (days).

FIG. 22 is a graph showing the effect of nitric oxide and/or glycolysis inhibitors on platelet swirl (0-3 units) of platelets as a function of storage time (days).

FIG. 23 is a graph showing the effect of nitric oxide and/or glycolysis inhibitors on platelet ATP levels (μM/1×10¹¹ platelets) as a function of storage time (days).

FIG. 24 is a graph showing the effect of nitric oxide and/or glycolysis inhibitors on % ESC of platelets as a function of storage time (days).

FIG. 25 is a graph showing the effect of nitric oxide on BVDV inactivation (log/ml) as a function of energy (J/cm²).

FIG. 26 is a graph showing the effect of nitric oxide on BVDV inactivation (log/ml) as a function of time (minutes).

### DETAILED DESCRIPTION OF THE INVENTION

As used herein, “adding nitric oxide to a fluid” refers to increasing the amount of nitric oxide within a fluid. In the practice of this invention nitric oxide may be added to a fluid by any method known in the art. Methods for adding nitric oxide to a fluid include, but are not limited to, adding liquids, solids, or gases containing nitric oxide and adding nitric oxide generators.

Nitric oxide generators are chemicals that are able to react, directly or indirectly, to produce nitric oxide. Nitric oxide generators may react with components already in a fluid to produce nitric oxide, or they may require the addition of one or more different nitric oxide generators to the fluid, with which they may react to produce nitric oxide. Nitric oxide generators that do not require the addition of one or more different nitric oxide generators are nitric oxide donors. Nitric oxide donors are well known in the art (Bauer et al., (1995) Advances in Pharmacology 34:361 and U.S. Pat. No. 6,232,434) and are available for purchase from companies such as Cayman Chemical, Ann Arbor, Mich. Nitric oxide donors include, but are not limited to L-arginine, N-acetyl-L-cysteine, DEA-NO, DETA-NO, DETA-NONOate, PAPA-NO, sodium nitroprusside, and nitroglycerine. Liquids containing nitric oxide include, but are not limited to liquids comprising two nitric oxide generators combined in a fluid to produce nitric oxide, saline in which nitric oxide gas has been bubbled, and nitric oxide-saturated water.

As used herein, “nitric oxide source” refers to an object that is capable of providing nitric oxide, e.g. a canister of nitric oxide gas. As used herein, “second nitric oxide source” refers to a nitric oxide source that may be the same as or different from the first nitric oxide source. As used herein, “nitric oxide gas” refers to a gas phase comprising nitric oxide, including but not limited to pure nitric oxide in gas phase, a gas comprising diluted pure nitric oxide, gas comprising or consisting of nitric oxide and nitrogen, gas comprising or consisting of nitric oxide and air, and gas comprising or consisting of nitric oxide and oxygen. As used herein, “air” refers to atmosphere at the surface of the earth, which is approximately 20% oxygen and 80% nitrogen. As used herein, “dissolved nitric oxide content” refers to the amount of nitric oxide dissolved in a fluid. e.g. As used herein, “oxygen source” refers to an object that is capable of providing oxygen, e.g. a canister of oxygen gas.

As used herein, “L-arginine source” refers to an object that is capable of providing L-arginine, e.g. a test tube containing an L-arginine solution. As used herein, “N-acetyl-cysteine source” refers to an object that is capable of providing N-acetyl-cysteine, e.g. a test tube containing an N-acetyl-cysteine solution. As used herein, “glycolysis inhibitor source ” refers to an object that is capable of providing glycolysis inhibitor e.g. a test tube containing a glycolysis inhibitor in solution.

As used herein, “pathogen” refers to an individual pathogenic organism of one species, a plurality of such organisms of one species, or a plurality of pathogenic organisms of two or more species. As used herein, “increase pathogen inactivation” with respect to the effects of a procedure described herein refers to inactivation of a greater quantity of pathogens after using the procedure than in the absence of the procedure.

As used herein, “ambient conditions” refers to about 1 atmosphere pressure and about 25° C.

As used herein, “endogenously-based derivative photosensitizer” refers to photosensitizers including but not limited to 7,8-dimethyl-10-ribityl isoalloxazine, 7,8-dimethylalloxazine, 7,8,10-trimethylisoalloxazine, alloxazine mononucleotide, isoalloxazine-adenosine dinucleotide, and neutralization-effective, substantially non-toxic amount of a microorganism neutralizers of formula:

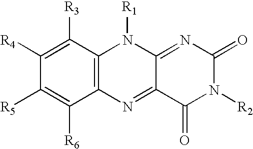

wherein R1, R2, R3, R4, R5 and R6 are, independently from one another, selected from the group consisting of hydrogen, optionally substituted hydrocarbyl, alcohol, amine, polyamine, sulfate, phosphate, halogen selected from the group consisting of chlorine, bromine and iodine, salts of the foregoing, and —NR^{a}—(C^{b}R^{c})ₙ—X wherein X is a halogen selected from the group consisting of chlorine, bromine and iodine, R^{a}, R^{b} and R^{c} are, independently of each other, selected from the group consisting of hydrogen, optionally substituted hydrocarbyl, and halogen selected from the group consisting of chlorine, bromine and iodine, and n is an integer from 0 to 20.

As used herein “photosensitizer” refers to any compound which absorbs radiation of one or more defined wavelengths and subsequently utilizes the absorbed energy to carry out a chemical process. Photosensitizers of this invention may include compounds which preferentially adsorb to nucleic acids, thus focusing their photodynamic effect upon microorganisms and viruses with little or no effect upon accompanying cells or proteins. Other photosensitizers of this invention are also useful, such as those using singlet oxygen-dependent mechanisms.

As used herein, “glycolysis inhibitor” refers to compositions that interfere with the biochemical pathway of glycolysis. 2-deoxy-D-glucose is an example of a glycolysis inhibitor.

Pathogen kill using riboflavin and related photosensitizer compounds occurs upon photoactivation via singlet oxygen damage, or via binding of the photosensitizer to nucleic acids of the pathogen, thereby disrupting the ability of the pathogen to function and reproduce, or both. Photosensitizer may not be recycled and reused in the system when irreversible reactions occur (such as the conversion of riboflavin to lumichrome which does not respond to visible light). If oxygen is present in the system, however, riboflavin may be sent down the singlet oxygen pathway, whereby singlet oxygen is produced and the photosensitizer molecule is recycled and returned to its ground state where it is again available for irradiation to produce the triplet state and can again react with oxygen to form more singlet oxygen. Alternatively, it can bind to proteins in the system. The formation of these riboflavin-protein adducts also removes riboflavin from the system and reduces the efficiency of the pathogen inactivation progress.

When oxygen is depleted in the system, irreversible reactions are favored: (1) reactions converting the photosensitizer to compounds which are not photoactive under visible light; and (2) binding reactions to proteins such that the photosensitizer is not free to effect further pathogen kill, both of which reactions remove photosensitizer as an active component of the system. More effective pathogen kill is therefore achieved when oxygen is added to favor reversible reactions in which the photosensitizer is recycled. Irradiating the fluid causes riboflavin to consume oxygen. When oxygen is not present in sufficient quantity, the irradiation process will consume the photoactivator. Addition of oxygen is therefore required to maintain production of singlet oxygen. Reducing light intensity (for low oxygen environments) helps to prevent the conversion of riboflavin to a form which is no longer capable of making singlet oxygen. Optimal systems of this invention are those providing maximum recycling of photoactivator.

As used herein, “quencher source” refers to an object that is capable of providing quencher, e.g. a test tube of quencher. Quenchers quench side chemical reactions generated by a photosensitizer and light. Quenchers include antioxidants or other agents to prevent damage to desired fluid components or to improve the rate of inactivation of microorganisms and are exemplified by adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, trolox, glycerol, vitamin E and mixtures thereof.

As used herein, “agitator” refers to an apparatus which can. agitate, e.g. shake or rotate, the container containing the product to be irradiated, such as the Helmer platelet incubator/agitator (Helmer Company, Noblesville, Ind.).

As used herein, “vital quality” refers to a parameter of a blood component that can be measured to assess quality, including indicators of cell quality. Indicators of blood component quality (vital qualities) include but are not limited to activation, hypotonic shock response, lactate production, glucose consumption, pH, platelet swirl, platelet aggregation, oxygen consumption, carbon dioxide production, cell count, and extent of shape change (ESC). As used herein, to “improve a vital quality of a blood component” refers to improving a parameter of a blood component that can be measured to assess quality, including the previously mentioned parameters. An improved blood component provides better results when utilized, for example, to treat patients.

GMP-140, also known as P-selectin, measures activation. When cells are activated, P-selectin appears on the surface of the cells. When P-selectin is high, the cells are activated. Platelet cells must retain the ability to activate when they are taken out of long-term storage to function normally for transfusion purposes. Cells need to be activated in vivo, so premature activation in vitro needs to be prevented. Specifications setting limits for values of certain vital qualities of blood components for use in treating patients are set by the Food and Drug Administration (FDA) of the U.S. (See Circular of Information for the Use of Human Blood and Blood Components or http://www.fda.gov/cber/gdlns/circbld.pdf ). The specified upper limit for activation in vitro is 60%. During cell metabolism, cells consume glucose and make two lactose molecules, which lowers the pH. The specified lower limit for pH of the surrounding fluid is 6.2. A fixed amount of glucose is provided to cells in storage. If the cells use up the glucose too quickly, they will die. A slower consumption of glucose is better, resulting in less lactose production, and maintenance of a pH above 6.2. Glucose consumption, lactose production, and pH indicators of cell quality are measured in rate as well as absolute change. Hypotonic shock response (HSR) measures the ability of the cells to respond to osmotic shock after about a ten-minute recovery period. Percent HSR measures the percentage of cells that are able to recover in about ten minutes. The specified lower limit for HSR is about 36%. Platelet swirl is a subjective, qualitative indicator of cell quality. When a blood bag is squeezed, healthy cells will swirl, creating a pattern which can be observed by the light reflecting off and through the cells. Platelet swirl is scored on a scale of from zero to three, with three being the healthiest. The quantity of cells swirling and the strength of the swirl are two characteristics that are considered. The extent of shape change (ESC) is the extent to which blood component cells are able to change shape when contacted with an agonist. Percent ESC measures extent of shape change. The specified lower limit for ESC is 10%. Platelet ATP levels indicate the amount of ATP in a solution.

Three platelet activity parameters that are measured to determine whether platelets have retained their functional ability after storage are: platelet number, hypotonic stress response, and aggregation, as induced by collagen in combination with adenosine diphosphate (ADP).

Hypotonic stress response is an assay used to determine if platelets have retained metabolic viability. This assay is a photometric measurement of the platelets' ability to overcome the addition of a hypotonic solution. This activity reflects cell function (i.e., ability to maintain a functional membrane water pump) and is indicative of platelet recovery following storage. Hypotonic stress response has been demonstrated to be an important indicator of platelets' ability to survive in circulation following transfusion. Consequently, hypotonic stress response represents an important parameter for evaluating platelet biochemistry following storage.

Potential for aggregation is another vital quality that indicates whether blood platelets have maintained their functional integrity during storage. This potential is measured by using ADP and collagen to induce aggregation. An agonist is an agent that binds to a receptor and initiates a certain response. In an agonist-induced aggregation, aggregation or clumping is the response to the agonist. The agonists ADP and collagen are used to induce aggregation to determine if platelets have retained their ability to aggregate. In addition, when performing aggregation response tests, one can detect the presence of spontaneous aggregation, that is the platelets adhering to each other without the addition of an agonist. The occurrence of spontaneous aggregation has been correlated with removal of platelets from circulation, indicating the platelets have short survival times.

This invention provides methods for decontaminating a fluid comprising a blood component and decontamination systems for performing such decontamination methods. The decontamination methods utilize a photoradiation pathogen inactivation process in which blood components are preserved by methods using nitric oxide, quencher, and/or glycolysis inhibitor. Vital cell qualities of the blood component are assayed to quantitate preservation of biological activity of the blood component after pathogen inactivation. Nitric oxide is provided as nitric oxide gas or through the use of one or more nitric oxide generators. Nitric oxide generators useful in the practice of this invention include L-arginine and N-acetyl-cysteine.

In the process of this invention, the concentration of nitric oxide gas used is an amount sufficient to improve a vital quality of the blood component, improve platelet storage life, and/or improve pathogen inactivation, but not so much as to interfere with these processes or be toxic to the blood components being treated; and is generally about 1200 ppm or less. In an embodiment of this invention, the energy delivered to the fluid is an amount sufficient to activate the photosensitizer, e.g., between about 5 J/cm² and about 10 J/cm². In an embodiment of this invention, the total time of photoradiation is sufficient to substantially inactivate the pathogens, e.g., between about six and about twelve minutes. To “substantially inactive pathogens” means to reduce their ability to reproduce, preferably by killing them, to levels in a blood component such that the blood component may be safely administered to a patient. The photoradiation is preferably within the ultraviolet range or the visible range. The wavelength of the photoradiation is preferably about 280 nm to about 700 nm. More preferably the wavelength of the photoradiation is between about 280 nm and about 320 nm or between about 415 nm and about 450 nm. In a specific embodiment, the wavelength is 419 nm. In another specific embodiment, the wavelength is 320 nm. In an embodiment of this invention, before photoradiation, the fluid is mixed by mixing and/or shaking at a speed between about 70 cpm and about 150 cpm, or between about 120 cpm and about 135 cpm. The mixing and shaking may performed in any motion known to the art. In an embodiment of this invention, the mixing and shaking comprises a to and fro motion. In an embodiment of this invention, one or more of the light sources may move in a coordinated manner with the movement of the mixing. Mixing may enable the majority of the photosensitizer and fluid contained within the container to be exposed to the light emitted from each of the discrete radiation sources by continually replacing the exposed fluid at the light-fluid interface with fluid from other parts of the bag not yet exposed to the light. Such mixing continually brings to the surface new fluid to be exposed to light.

In the practice of this invention, the concentration of L-arginine is sufficient to generate sufficient nitric oxide to enhance a vital quality of a blood component being treated, but not so much as to be toxic to the blood component or interfere with pathogen inactivation, e.g. between about 25 μM and about 3 mM. In one specific embodiment of this invention, the concentration of L-arginine is at least about 25 μM. In another specific embodiment of this invention, the concentration of L-arginine is between about 100 μM and about 500 μM. Preferably, the concentration of L-arginine is about 100 μM.

In the practice of this invention, the concentration of N-acetyl-cysteine is sufficient to generate sufficient nitric oxide to enhance a vital quality of a blood component being treated, but not so much as to be toxic to the blood component or interfere with pathogen inactivation, e.g., between about 25 μM and about 3 mM. In one specific embodiment of this invention, the concentration of N-acetyl-cysteine is at least about 25 μM. In another specific embodiment of this invention, the concentration of N-acetyl-cysteine is between about 100 μM and about 500 μM. Preferably, the concentration of N-acetyl-cysteine is about 100 μM.

In the practice of this invention, the concentration of 2-deoxy-D-glucose is sufficient to inhibit glycolysis and increase storage life of blood cellular blood components being treated, but not so much as to be toxic to such cellular blood components, e.g., between about 1 mM and about 10 mM. In an embodiment of this invention, the concentration of 2-deoxy-D-glucose is at least about 1 mM. Preferably, the concentration of 2-deoxy-D-glucose is about 10 mM.

In the practice of this invention, at least one vital quality of the nitric oxide-contacted and photoradiated blood component is improved. All indicators of cell quality known in the art may be measured. In an embodiment of this invention, indicators of cell quality measured in the practice of this invention include activation, hypotonic shock response, lactate production, glucose consumption, pH, platelet swirl, and platelet aggregation. Indicators of cell quality are typically measured on Days 1, 3, 5, and/or 7 after photoradiation.

In the practice of this invention, preferably blood component activation, is decreased, as compared to blood component activation in a similar pathogen inactivation process that does not use nitric oxide. Preferably activation is measured by GMP-140, but any method known in the art may be used. Activation, is preferably decreased by at least about 3%, more preferably by at least about 25%, and more preferably by at least about 50%. In an embodiment of this invention, activation is decreased by an amount between about 25% and about 75%.

In the practice of this invention, preferably hypotonic shock response (HSR) is increased, as compared to HSR in a similar pathogen inactivation process that does not use nitric oxide. HSR is preferably increased by at least about 6%, more preferably by at least about 25%, more preferably by at least about 50%, more preferably by at least about 75%, and more preferably by at least about 100%. In an embodiment of this invention, HSR is increased by an amount between about 25% and about 125% as measured on Day 5 after photoradiation. In an embodiment of this invention, HSR is increased by an amount between about 500% and about 3000% as measured on Day 7 after photoradiation.

In the practice of this invention, preferably platelet swirl is increased, as compared to platelet swirl in a similar pathogen inactivation process that does not use nitric oxide. Platelet swirl is preferably increased by at least about 7%, more preferably by at least about 20%. In an embodiment of this invention, platelet swirl is increased by an amount between about 20% and about 100% as measured on Day 5 after photoradiation.

In the practice of this invention, preferably the pH of the fluid and the rate of pH decrease are decreased, as compared to pH and rate of pH decrease in a similar pathogen inactivation process that does not use nitric oxide. The decreased rate of pH increase can also be measured, if pH increases. The rate of pH decrease is preferably decreased by at least about 2%, more preferably by at least about 20%, and more preferably by at least about 35%. In an embodiment of this invention, the rate of pH decrease is decreased by an amount between about 2% and about 50%. The pH is preferably decreased by at least about 0.1 units, more preferably by at least about 0.2, and more preferably by at least about 0.35. In an embodiment of this invention, the pH is decreased by an amount between about 0.1 and about 0.75. In an embodiment of this invention, the rate of pH decrease is preferably decreased by an amount between about 15% and about 25% on Day 5.

In the practice of this invention, preferably the rate of lactate production by blood components and the amount of lactate produced are decreased, as compared to rate of lactate production and amount of lactate produced in a similar pathogen inactivation process that does not use nitric oxide. The rate of lactate production is preferably decreased by at least about 3%, more preferably by at least about 10%, more preferably by at least about 30% and more preferably by at least about 50%. In an embodiment of this invention, the rate of lactate production is decreased by an amount between about 10% and about 65%. The amount of lactate produced is preferably decreased by at least about 0.1, more preferably by at least about 15%, and more preferably by at least about 30%. In an embodiment of this invention, the amount of lactate produced is decreased by an amount between about 15% and about 50%. In an embodiment of this invention, the rate of lactate production is decreased by an amount between about 10% and about 45% on Day 5.

In the practice of this invention, preferably the rate of glucose consumption by blood components and the amount of glucose consumed are decreased, as compared to rate of glucose consumption and amount of glucose consumed in a similar pathogen inactivation process that does not use nitric oxide. Glucose consumption is measured by quantitating the amount of glucose remaining in the fluid at a time point. The decrease in glucose consumption is measured here as the increase in the amount of glucose remaining in the fluid. The decrease in the rate of glucose consumption is calculated by measuring the amount of glucose remaining in a fluid at two or more time points after photoradiation by a method of this invention, subtracting the amount of glucose left at time 1 (the earlier time) from the amount at time 2 (the later time), dividing by time 1 subtracted from time 2 to calculate a rate of glucose consumption; and then comparing the calculated rate of glucose consumption to a rate of glucose consumption for a control sample, such as a sample photoradiated without added nitric oxide, quencher, and glycolysis inhibitor. When the rate of glucose consumption is decreased, more glucose is left in the fluid at a selected time point, on average, in comparison to a control sample. The rate of glucose consumption is preferably decreased by at least about 3%, more preferably by at least about 25%, and more preferably by at least about 75%. In an embodiment of this invention, the rate of glucose consumption is decreased by an amount between about 25% and about 150%. The amount of glucose consumed is preferably decreased by at least about 40%. In an embodiment of this invention, the rate of glucose consumption is decreased by an amount between about 10% and about 150%, more preferably between about 25% and about 90% on Day 3.

In the practice of this invention, preferably the extent of cell shape (ESC) of the blood component is increased, as compared to the ESC of blood components that have undergone a pathogen inactivation process that does not use nitric oxide. In an embodiment of this invention, the ESC is increased by an amount between about 2% and about 25%. In an embodiment of this invention, the ESC is increased by at least about 2%. In an embodiment of this invention, the ESC is increased by at least about 25%.

As used herein, “an amount sufficient to improve storage life” refers to an amount of nitric oxide that increases a vital cell quality of the blood component that limits storage life of that blood component. Vital cell qualities that determine allowed storage life of blood components are determined by the U.S. FDA ( See Circular of Information for the Use of Human Blood and Blood Components or http://www.fda.gov/cber/gdlns/circbld.pdf ). To increase storage life of a blood component, the vital cell quality that is limiting the storage life of that blood component must be improved. Additional vital cell qualities may be improved as well. In the practice of this invention, when about 150 ml of between about 25 ppm and about 500 ppm nitric oxide gas is added to between about 275 ml and about 300 ml plasma of about 90% carry-over, the vital quality of blood component activation may be decreased by at least about 3%, or the vital quality of HSR may be increased by at least about 6%, or the vital quality of platelet swirl may be increased by at least about 7%, or the vital quality of pH of the fluid containing the blood component may be decreased by at least about 0.1 pH units, or the vital quality of rate of lactate production may be decreased by at least about 10%, or the vital quality of rate of glucose consumption may be decreased by at least about 3%. In an embodiment of this invention, storing said blood component for a period greater than five days is between more than five days and about seven days.

As used herein, “substantially inactivated” refers to fluid being decontaminated of pathogens to a level sufficient to meet requirements for intravenous introduction into a human body. In the practice of this invention, pathogens which may be present in a fluid to be treated are substantially inactivated. In an embodiment of this invention, pathogens which may be present in a fluid to be treated are inactivated by at least about 35%, by at least about 90%, by at least about 95, by at least about 99%, by at least about 99.9% or by at least about 100%. In the practice of this invention, pathogen inactivation is preferably increased, as compared to a similar pathogen inactivation process that does not use nitric oxide. In the practice of this invention, preferably pathogen inactivation is at least not substantially decreased, as compared to a similar pathogen inactivation process that does not use nitric oxide.

This invention provides a method for treating a fluid comprising a blood component to inactivate pathogens which may be present therein, comprising the steps of:

(a) adding an inactivation-effective, substantially non-toxic amount of an endogenous photosensitizer or endogenously-based derivative photosensitizer to said fluid;

(b) adding nitric oxide to said fluid to increase dissolved nitric oxide content of said fluid in an amount sufficient to improve a vital quality of said blood component; and

(c) exposing said fluid to photoradiation of sufficient energy to activate the photosensitizer, for a sufficient time to substantially inactivate said pathogens.

The dissolved nitric oxide content of said fluid may also be in an amount sufficient to increase pathogen inactivation. In an embodiment of this invention, adding nitric oxide is performed by a method selected from the group consisting of adding nitric oxide gas, adding a nitric oxide generator, adding L-arginine, and adding N-acetyl-cysteine. The method may also comprise increasing the dissolved oxygen content of said fluid to an amount sufficient to enhance a reaction of the photosensitizer in which singlet oxygen and reactive oxygen species (ROS) are formed. The method may also comprise adding a quencher to said fluid. The method may also comprise adding a glycolysis inhibitor to said fluid. The glycolysis inhibitor may be 2-deoxy-D-glucose. The blood component comprises a component selected from the group consisting of plasma, platelets, red blood cells, white blood cells, and plasma proteins. Preferably, the blood component comprises platelets. The fluid may comprise platelets in a solution comprising plasma and storage solution, such as platelets (in plasma) and storage solution at a ratio between about 20:80 and about 90:10 platelets:storage solution. In an embodiment of this invention, the method is performed in a blood product collection bag., such as those useful with the Trima® and Spectra™ apheresis systems manufactured by Gambro BCT, Inc. (Lakewood, Colo., USA). In an embodiment of this invention, the photosensitizer is selected from the group consisting of endogenous isoalloxazines and isoalloxazine derivative photosensitizers, such as 7,8-dimethyl-10-ribityl isoalloxazine, 7,8-dimethylalloxazine, 7,8,10-trimethylisoalloxazine, alloxazine mononucleotide, and isoalloxazine-adenosine dinucleotide. Preferably, the photosensitizer is riboflavin. In one embodiment the photosensitizer is at about 50-micromolar concentration.

When adding nitric oxide comprises adding nitric oxide gas, between about 25 ml and about 1200 ml nitric oxide gas should be added, e.g., about 150 ml nitric oxide gas. When amounts of gas are referred to herein by measurements of volume, such volumes are those existing at one atmosphere of pressure. In an embodiment of this invention, the nitric oxide gas added is of a concentration between about 10 ppm and about 1000 ppm nitric oxide gas. In one embodiment between about 25 ppm and about 500 ppm nitric oxide gas is added to the fluid. In a further embodiment about 25 ppm and about 50 ppm nitric oxide gas is added to the fluid. In an embodiment of this invention, the nitric oxide gas is diluted with nitrogen. When a gas is referred to herein as being in a “balance” of another gas, it means that the container holding the gas contains that gas, and that the balance (remainder) of the gas in the container is the other gas. For example, 25 ppm of nitric oxide in a nitrogen balance, is 25 ppm of nitric oxide in a container at one atmosphere of pressure in which the “balance” (remainder) of the gas in the container is nitrogen The nitric oxide gas would be in a nitrogen balance at one atmosphere of pressure when the nitric oxide gas contributes a portion of the pressure and the nitrogen gas contributes the remaining pressure required to reach one atmosphere. Air is about 20% oxygen and about 80% nitrogen. In an embodiment of this invention, the nitric oxide gas is supplied in a concentrated form in a nitrogen balance, and is diluted in an air balance, in about a 20% oxygen and about an 80% nitrogen balance. To make 150 ml of 25 ppm nitric oxide gas in an air balance, 15 ml of 500 ppm nitric oxide gas in a nitrogen balance could be combined with 135 ml of air.

When adding nitric oxide comprises adding nitric oxide generator, L-arginine, N-acetyl-cysteine, and/or any another nitric oxide generator known in the art may be added.

L-arginine may act, in part, by stimulation of the cyclic GMP (cGMP) pathway. Other compositions that stimulate the cGMP pathway, including lipophilic analogues of cGMP and cAMP, are useful in the practice of this invention. Examples of analogues which may be used in this invention are dibutyryl guanosine monophosphate (cGMP analogue) and dibutyryl adenosine monophosphate (cAMP analogue) (available from Sigma, St. Louis, Mo.)

N-acetyl-cysteine is preferably added as N-acetyl-L-cysteine.

This invention also provides blood products decontaminated by the method of this invention.

The pathogens which may be present in the fluid and decontaminated by the processes of this invention typically include those selected from the group consisting of extracellular and intracellular viruses, bacteria, bacteriophages, fungi, blood-transmitted parasites, and protozoa, and mixtures of any two or more of the foregoing. If one of the pathogens is a virus, it may be selected from the group consisting of human immunodeficiency virus (HIV), hepatitis A, B and C viruses, sindbis virus, cytomegalovirus, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, and Epstein-Barr virus, bovine viral diarrhea virus, pseudorabies, West Nile virus, and mixtures of any two or more of the foregoing. If one of the pathogens is a bacteriophage, it may be selected from the group consisting of Φ X174, Φ6, λ, R17, T₄, and T₂, and mixtures of any two or more of the foregoing. If one of the pathogens is a bacterium, it may be selected from the group consisting of P. aeruginosa, S. aureus, S. epidermidis, E. coli, K. pneumoniae, E. faecalis, B. subtilis, S. pneumoniae, S. pyrogenes, S. viridans, B. cereus, E. aerogenes, propionabacter, C. perfringes, E. cloacae, P. mirabilis, S. cholerasuis, S. liqfifaciens, S. mitis, Y entercolitica, P. fluorescens, S. enteritidis, C. freundii, and S. marcescens, and mixtures of any two or more of the foregoing. In an embodiment of this invention, if one of the pathogens is a protozoa, it may be P. falciparum.

It has been observed that a side effect of a pathogen reduction process is that when platelets are subjected to UV light, the mitochondria of the platelets have a greater chance of suffering at least some damage than when they have been subjected to visible light. If mitochondrial function is suppressed by UV light, platelets are unable to create ATP (energy) through aerobic respiration. If platelets are unable to create energy through aerobic respiration, they create energy through the glycolysis pathway. One metabolite produced by the glycolysis pathway is lactic acid. Lactic acid buildup within cells causes the pH of the surrounding solution to drop. Such a drop in pH causes decreased cell quality during storage.

One way to prevent this pH drop and subsequent drop in cell quality is to prevent the buildup of lactic acid. This may be done by using an agent or agents which block or slow glycolysis. 2-deoxy-D-glucose (2-DOG) is one such agent which slows the rate of glycolysis by inhibiting enzymatic processes within the glycolytic chain and may be added to the fluid to be pathogen reduced in the manner described above. Glycolysis inhibitors include but are not limited to 2-deoxy-D-glucose, xylose, arabinose and lyxose.

In one embodiment, 2-deoxy-D-glucose is added to a fluid containing a blood component comprising platelets before the platelets are subjected to a pathogen inactivation procedure, in an amount that improves blood component quality during storage. In a preferred embodiment, 2-deoxy-D-glucose is added to the fluid at a concentration of 1 mM to 10 mM. In an alternative embodiment, 2-deoxy-D-glucose is added to platelets after a pathogen reduction procedure in such an amount, to aid in storage of the pathogen reduced platelets.

This invention provides a biological composition comprising:

(a) a fluid;

(b) an inactivation-effective, substantially non-toxic amount of an endogenous photosensitizer or endogenously-based derivative photosensitizer; and

(c) dissolved nitric oxide in an amount greater than would be present under an air atmosphere at ambient conditions without mixing.

The composition may also comprise dissolved oxygen in an amount greater than would be present under an air atmosphere at ambient conditions without mixing. The composition may also comprise a quencher. The composition may also comprise a glycolysis inhibitor. In an embodiment of this invention, the composition also comprises one or more blood components selected from the group consisting of plasma, red blood cells, white blood cells, platelets and plasma proteins. Preferably the blood component comprises platelets. In an embodiment of this invention, the composition comprises platelets and storage solution. The composition may also comprise pathogens to be inactivated. When the composition comprises pathogens, the pathogens are typically those selected from the group consisting of extracellular and intracellular viruses, bacteria, bacteriophages, fungi, blood-transmitted parasites, and protozoa, and mixtures of any two or more of the foregoing. Preferably the pathogens have been substantially inactivated. In an embodiment of this invention, this composition is in a blood component bag. In an embodiment of this invention, the blood component bag comprises between about 100 and about 600 ml of the composition. In an embodiment of this invention, the blood component bag also comprises air. In an embodiment of this invention, the blood component bag also comprises more oxygen than is present in air. In an embodiment of this invention, the blood component bag also comprises more nitric oxide than is present in air. In an embodiment of this invention, the blood component bag also comprises more oxygen and more nitric oxide than is present in air.

This invention provides a decontamination system for a fluid comprising:

(a) a leak-proof transparent or translucent container for the fluid, wherein said container comprises one or more inlets;

(b) a photosensitizer source for providing photosensitizer to said container, said photosensitizer source being connectible to an inlet of said container;

(c) a nitric oxide source connectible to an inlet of said container for providing nitric oxide to said container; and

(d) a photoirradiator, i.e., a source of visible or ultraviolet radiation, for irradiating said container.

The container is preferably a blood collection bag or a blood product bag. In an embodiment of this invention, the decontamination system also comprises an oxygen source connectible to an inlet of said container for providing oxygen to said container. In an embodiment of this invention, the decontamination system also comprises a quencher source for providing quencher to said container, said quencher source being connectible to an inlet of said container. In an embodiment of this invention, the decontamination system also comprises a glycolysis inhibitor source for providing glycolysis inhibitor to said container, said glycolysis inhibitor source being connectible to an inlet of said container. In an embodiment of this invention, the decontamination system also comprises an agitator for agitating said container. In an embodiment of this invention, the decontamination system also comprises a sterile barrier between said nitric oxide source and said container. In an embodiment of this invention, the transparent or translucent container is a blood product bag. In an embodiment of this invention, the decontamination system's blood product bag contains a fluid comprising a blood component selected from the group consisting of plasma, platelets, red blood cells, white blood cells, and plasma proteins. In an embodiment of this invention, the blood component is platelets. In an embodiment of this invention, the fluid comprises platelets (in plasma) and storage solution at a ratio between about 20:80 and about 90:10 platelets:storage solution. In an embodiment of this invention, in the decontamination system the irradiator is a visible light irradiator. In an embodiment of this invention, in the decontamination system the agitator is a mixer/shaker. In an embodiment of this invention, the decontamination system also comprises a scale for weighing said container. In an embodiment of this invention, the decontamination system also comprises a bar-coded label for said container and a scanner for reading said bar-coded label. In an embodiment of this invention, the decontamination system also comprises a computer processor for receiving, correlating and storing data comprising data selected from the group consisting of data identifying said container, the weight of said container, the fact that said container has been irradiated, and the protocol utilized to irradiate said container. In an embodiment of this invention, the decontamination system also comprises at least one apparatus for maintaining temperature of said fluid and/or at least one apparatus for agitating said container before and/or after irradiation. In an embodiment of this invention, there is more than one nitric oxide source. In an embodiment of this invention, the nitric oxide source comprises a source of nitric oxide gas and/or one or more sources of nitric oxide generator. In an embodiment of this invention, the nitric oxide source is selected from the group consisting of sources of nitric oxide gas, sources of L-arginine, and sources of N-acetyl-cysteine.

In decontamination systems of this invention, the photoradiation source may be connected to the photopermeable container for the fluid by means of a light guide such as a light channel or fiber optic tube which prevents scattering of the light between the source and the container for the fluid, and more importantly, prevents substantial heating of the fluid within the container. Direct exposure to the light source may raise temperatures as much as 10 to 15° C., especially when the amount of fluid exposed to the light is small, which can cause denaturization of blood components. Use of the light guide keeps any heating to less than about 2° C. The method may also include the use of temperature sensors and cooling mechanisms where necessary to keep the temperature below temperatures at which desired proteins in the fluid are damaged. Preferably, the temperature is kept between about 0° C. and about 45° C., more preferably between about 4° C. and about 37° C., and most preferably about 22° C. The photoradiation source may be a simple lamp or may consist of multiple lamps radiating at differing wavelengths. The photoradiation source should be capable of delivering from about 1 J/cm² to at least about 120 J/cm².

This invention provides a method for making a decontamination system comprising the steps of:

(a) providing a set of components comprising:

(i) a leak-proof transparent or translucent container for a fluid wherein said container comprises one or more inlets;

(ii) a photosensitizer source for providing photosensitizer to said container, said photosensitizer source comprising an outlet connectible to one of said inlets of said container;

(iii) a nitric oxide source for providing nitric oxide to said container, said nitric oxide source comprising an outlet connectible to one of said inlets of said container; and

(iv) a photoirradiator for irradiating said container; and

(b) assembling said components by connecting the outlet of said photosensitizer source to an inlet of said container, connecting the outlet of said nitric oxide source to an inlet of said container, and positioning the photoirradiator so as to irradiate said container.

The position of the photoirradiator will be determined by its heat output, the cooling capacity of the system, and the amount of energy need for the pathogen inactivation.

In an embodiment of this invention, the set of components also comprises an oxygen source for providing oxygen to said container, said oxygen source connectible to an inlet of said container. In an embodiment of this invention, the set of components also comprises a quencher source connectable to an inlet of said container. In an embodiment of this invention, the set of components also comprises a glycolysis inhibitor source connectable to an inlet of said container.

This invention also provides a method of increasing the storage life of platelets comprising the steps of:

(a) placing said platelets in a container larger than the volume of a solution containing said platelets; and

(b) adding nitric oxide to said fluid to increase dissolved nitric oxide content of said fluid in an amount sufficient to improve a vital quality of said blood component, wherein said vital quality is selected from the group of qualities consisting of activation, hypotonic shock response, lactate production, glucose consumption, pH, platelet swirl, and platelet aggregation;

(c) adding a photoactivator to said solution and irradiating said solution to activate said photoactivator; and

(d) storing said platelets.

In an embodiment of this invention, the method also comprises the step, performed before step (c), of dissolving an amount of oxygen in said solution greater than that which would be dissolved in said solution under an air atmosphere at ambient conditions without agitation. In an embodiment of this invention, the method also comprises removing substantially all gas phase from said container after step (c). In an embodiment of this invention, the method also comprises adding additional nitric oxide, e.g. after the gas phase has been removed, to said fluid after irradiation to increase the dissolved nitric oxide content of said fluid in an amount sufficient to further improve a vital quality of said blood component. In an embodiment of this invention, the method also comprises adding a quencher to said solution. The storage life of a blood component is increased by improving a vital quality of the blood component. Storage life for platelets can be increased from five to up to seven days using the methods and systems of this invention.

The methods of this invention do not include a step of deoxygenating the fluid in which pathogens are to be deactivated. At least one fluid utilized in the practice of this invention is not deoxygenated before the addition of nitric oxide. Oxygen may be present in the fluids used in the practice of this invention.

One aspect of this invention is directed to a method for maintaining the quality of blood components, in particular platelets, throughout a pathogen inactivation process. Another aspect of this invention is directed to increasing the percentage of inactivated pathogens in a solution containing at least one blood component, a photosensitizer, and one or more pathogens.

This invention further relates to the addition of nitric oxide (NO) to a solution containing riboflavin or other photosensitizer and a blood component. Oxygen (O₂) gas may optionally by added to the solution containing a blood component, a photosensitizer, and NO. The solution may then be irradiated using UV and/or visible light. After irradiation, the NO may be removed from the irradiated solution. Optionally, NO may be added to a solution containing a blood component to aid in the long-term storage of the blood component.

The invention may further include a bag or bags for storage of blood or blood components made of polymeric material capable of releasing NO over time into a solution contain contained therein.

Other components and methods are useful in the practice of this invention. All known blood components known in the art are useful in the practice of this invention. The blood component is preferably a blood component for medical use such as for transfusion. Preferably the blood component contains platelets. In an embodiment of this invention, the blood component contains at least 1×10⁵ platelets per microliter. Platelet solutions containing between about 25% and 90% plasma carry-over are useful in the practice of this invention. Fluids that may contain pathogens are useful in the practice of this invention. Blood components from any blood-containing animal are useful in the practice of this invention. In an embodiment of this invention, the blood component is from a human. Preferably the blood component is useful in the practice of veterinary or human medicine.

Photopermeable containers having fluid volumes between about 100 ml and 600 ml are useful in this invention, such as fluid volumes between about 275 ml and about 300 ml. In an embodiment of this invention, the container may be a 3L blood bag. Other blood bags, of smaller and larger sizes, and other amounts of fluid are useful. Other bag and fluid combinations can be compared to those used in the examples hereof using the measurement of aspect ratio, which equals the volume of fluid divided by the surface area of the bag. Aspect ratios between about 0.3 and 0.5 are useful in the practice of this invention, e.g., aspect ratios of between about 0.4 and about 0.43.

Materials which may be treated by the methods of this invention include any materials which are adequately permeable to photoradiation to provide sufficient light to achieve pathogen inactivation, or which can be suspended or dissolved in fluids which have such permeability to photoradiation. Examples of such materials are aqueous compositions containing biologically active proteins derived from blood or blood constituents. Whole blood, packed red cells, platelets and plasma (fresh or fresh frozen plasma) are exemplary of such blood constituents. In addition, therapeutic protein compositions containing proteins derived from blood, such as fluids containing biologically active protein useful in the treatment of medical disorders, e.g. factor VIII, Von Willebrand factor, factor IX, factor X, factor XI, Hageman factor, prothrombin, anti-thrombin III, fibronectin, plasminogen, plasma protein fraction, immune serum globulin, modified immune globulin, albumin, plasma growth hormone, somatomedin, plasminogen streptokinase complex, ceruloplasmin, transferrin, haptoglobin, antitrypsin and prekallikrein may be treated by the decontamination methods of this invention. The term “biologically active” means capable of effecting a change in a living organism or component thereof. The term “blood product” as used herein includes blood constituents and therapeutic protein compositions containing proteins derived from blood as defined above. Fluids containing biologically active proteins other than those derived from blood may also be treated by the methods of this invention.

Decontamination methods of this invention do not destroy the biological activity of fluid components other than microorganisms. “Substantially non-toxic” amounts of elements of this invention are those which do not destroy the biological activity of such fluid components other than microorganisms. As much biological activity of these components as possible is retained, although in certain instances, when the methods are optimized, some loss of biological activity, e.g., denaturization of protein components, must be balanced against effective decontamination of the fluid.

Nitric oxide may be added to a fluid using methods and compositions such as gases containing nitric oxide; fluids, solids, and liquids containing nitric oxide; and nitric oxide generators, including nitric oxide donors such as L-arginine and N-acetyl-cysteine. Nitric oxide generators may be natural or synthetic. The bag or other container containing the fluid, or an object inserted in the fluid, can be made with or coated with a nitric oxide generator. Nitric oxide gas includes gases comprising nitric oxide.

Other nitric oxide sources used in the practice of this invention should deliver an equivalent amount of dissolved nitric oxide to the fluid. In an embodiment of this invention, when nitric oxide gas that does not comprise oxygen is used in the practice of this invention, it is an oxygen-free nitrogen balance gas. A gas consisting of nitrogen balanced nitric oxide gas, has a specified amount of nitric oxide, e.g. 25 ppm nitric oxide, and the balance is nitrogen, at ambient atmospheric conditions, i.e., at 1 atm pressure. Nitric oxide gases that comprise nitric oxide and oxygen and/or nitrogen are useful in the practice of this invention. Nitric oxide gases containing a specified amount of nitric oxide, e.g., 25 ppm nitric oxide, wherein the balance is oxygen, are useful in the practice of this invention. Nitric oxide gases wherein the balance comprises between about 20% oxygen and about 100% oxygen are also useful. In an embodiment of this invention, the nitric oxide gas is an air balance. The amount of nitric oxide used in the practice of this invention enhances a vital quality of the blood components and may optionally enhance the inactivation of pathogens in the fluid, but does not substantially interfere with the biological activity of the blood component nor does it substantially interfere with inactivation of the pathogens which may be present in the fluid containing the blood component. Nitrogen oxide gas may be readily obtained (i.e., Scott Specialty Gases Inc. in Longmont, Colo.). Nitric oxide is available in a variety of concentrations, for example ranging from 1 ppm to 10,000 ppm. Because nitric oxide is a reactive gas, it must be bottled in an oxygen-free environment, such as in a nitrogen balance. Nitric oxide can be introduced into a closed bag via a syringe and a sterile barrier filter. Pure nitrogen gas can be used to dilute a nitric oxide gas.

The amount of photosensitizer to be mixed with the fluid will be an amount sufficient to adequately inactivate all microorganisms therein, but less than a toxic or insoluble amount. As taught herein, optimal concentrations for desired photosensitizers may be readily determined by those skilled in the art without undue experimentation. Photosensitizers useful in the practice of this invention include 7,8-dimethyl-10-ribityl isoalloxazine, 7,8-dimethylalloxazine, 7,8,10-trimethylisoalloxazine, alloxazine mononucleotide, isoalloxazine-adenosine dinucleotide, isoalloxazine derivative photosensitizers, and mixtures thereof, as well as the isoalloxazine analogs disclosed in U.S. patent application Ser. No. 09/777,727 and U.S. Pat. No. 6,268,120, incorporated by reference herein to the extent not inconsistent herewith. The fluid containing the photosensitizer is then exposed to photoradiation of the appropriate wavelength to activate the photosensitizer, using an amount of photoradiation sufficient to activate the photosensitizer as described above, but less than that which would cause non-specific damage to the biological components or substantially interfere with biological activity of other proteins present in the fluid. Addition of a photosensitizer may not be required for irradiation with UV light. Quenchers may be used to prevent unwanted side reactions generated by a photosensitizer and light. Quenchers quench the reactions that might cause unwanted damage to desired biological components.

Photoradiation to inactivate pathogens is performed by methods known in the art or by methods described in references included herein. An amount of energy is supplied to the fluid containing nitric oxide using photoradiation. Preferably the amount of energy supplied is sufficient to eradicate pathogens which may exist in the fluid, but also does not substantially interfere with the biological activity of the blood component(s) contained in the fluid. The biological activity of the blood component(s) in the fluid at least meets minimum standards for medical and veterinary use for standard storage times for the specific blood component, e.g., five or preferably seven days for platelets. Pathogen inactivation methods of this invention are described as using flux (energy) in units of joules (J) per unit area (cm²) per unit time (min). A time length of photoradiation is selected to accomplish delivering a total amount of energy selected to substantially inactivate pathogens in the fluid being treated. Some lamps useful for providing the required energy are VHO lights with a mercron ballast, T8 lights with an icecap ballast, or T8 lights with an icecap ballast and quartz attenuator. Photoradiation may be delivered continuously or in a segmented (interrupted) fashion.

Fluids may optionally be mixed before or during photoradiation. Mixing may enhance dissolution of nitric oxide or other components. Mixing may be performed using a shaker at mixing speed of between about 70 and about 150 cpm. In an embodiment of this invention, mixing occurs between about 120 cpm and about 135 cpm. Mixing is performed for about one to about ten minutes. Photoradiation is performed at a temperature that allows for inactivation of pathogens and does not interfere with the biological activity of blood components. Photoradiated fluids containing blood components are stored at temperatures known in the art for storing blood products.

In the practice of this invention, nitric oxide may be added to fluids to be decontaminated before and/or after photoradiation. In an embodiment of this invention, nitric oxide is added to a decontaminated fluid comprising a blood component, wherein the nitric oxide is in an amount sufficient to improve a vital quality of the blood component sufficient to increase the storage life of the blood component. In an embodiment of this invention, nitric oxide gas, L-arginine, and/or N-acetyl-cysteine is added to a fluid comprising a blood component that has been decontaminated by the methods of this invention.

### EXAMPLE 1

Effect of 500 ppm NO on PET UV Process using Plasma and E. coli

Pathogen eradication technology (PET) is a pathogen inactivation process that was carried out on a fluid comprising a blood component. A measured amount of fluid was placed in a translucent 3L Sengewald blood bag having no light-blocking labels. Other compositions such as pathogens, photosensitizer and/or quencher were optionally added. A measured concentration of a nitric oxide source was added to the bag. Other chemicals were optionally added, such as nitrogen and/or oxygen. Pathogen and/or cell count titers were optionally measured. PET was performed, using specified parameters: wavelength, energy, flux, light source, light configuration, mixing speed, temperature, and optionally mylar sheeting, and time of exposure. Pathogen titer and/or cell quality indicators were measured. The fluid was placed in blood storage bag, or optionally substantially all gas phase was removed from the original blood bag, and the photoradiated fluid was stored at about 22° C. for several days. Cell quality indicators and/or pathogen titers were optionally measured at any of one to seven days after PET.

Example 1 was performed using the parameters described in Table 1. Pathogen eradication was performed in a 3L Sengewald bag on 300 ml of plasma (90% carry-over) that was innoculated with E. coli. The bag also contained 150 ml of 500 ppm nitric oxide gas in a nitrogen balance, delivered through a sterile barrier filter, and 50 μM riboflavin. Mixing was performed before photoradiation at 120 cpm. Ultraviolet photoradiation of 320 nm wavelength delivered 6 J/cm² of energy, at about 30° C. Bacteria kill was monitored before photoradiation, at day 0 immediately after treatment, and at day 2. The results of pathogen inactivation immediately after treatment were compared to other experimental results using 5 J/cm² and 7 J/cm² UV irradiation energy, with illumination using similar conditions of VHO light bulbs with a mercron ballast, but with no NO and using only 278 ml of fluid. With E. coli, improvement of bacterial inactivation was seen using 500 ppm NO atmosphere during illumination at 6 J/cm², with both 5 and 7 J/cm² and with both VHO and T8 lights.

### EXAMPLE 2

Effect of 500 ppm NO on PET UV Process using Plasma and S. epidermidis

Example 2 was performed using the parameters described in Table 2. Treatment 2 was identical to treatment 1, except that S. epidermidis was used as the bacteria. With S. epidermidis improvement of bacterial inactivation was seen using 500 ppm NO atmosphere during illumination at 6 J/cm², with both 5 and 7 J/cm² and with VHO lights. There was no difference with the T8 lights.

### EXAMPLE 3

Effect of 500 ppm NO on PET Visible Light Process and CQ, using Plasma and BVDV

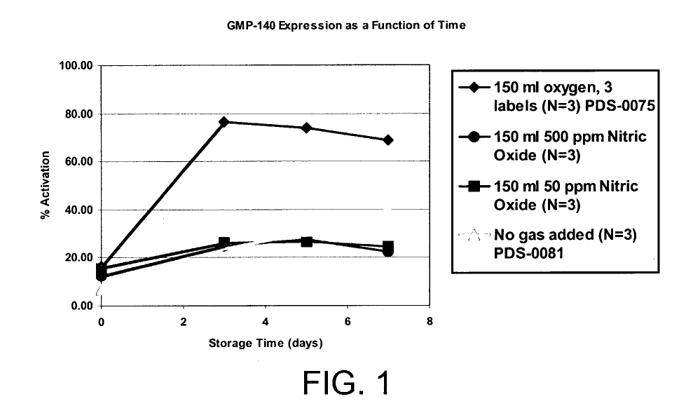

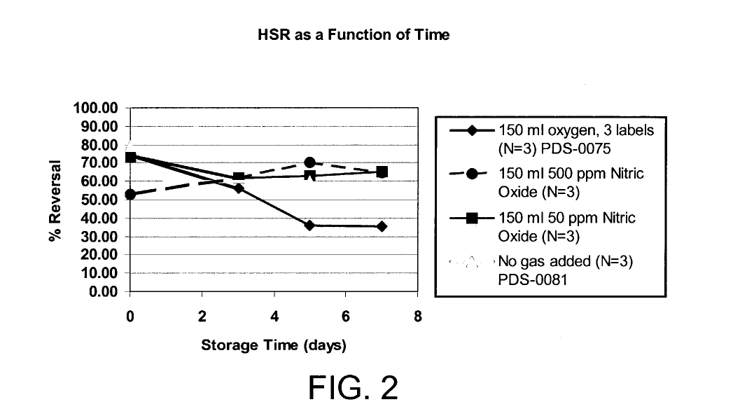

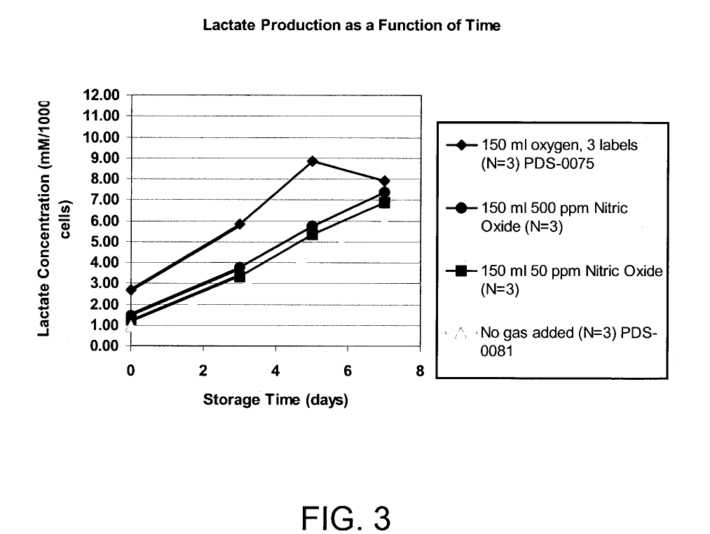

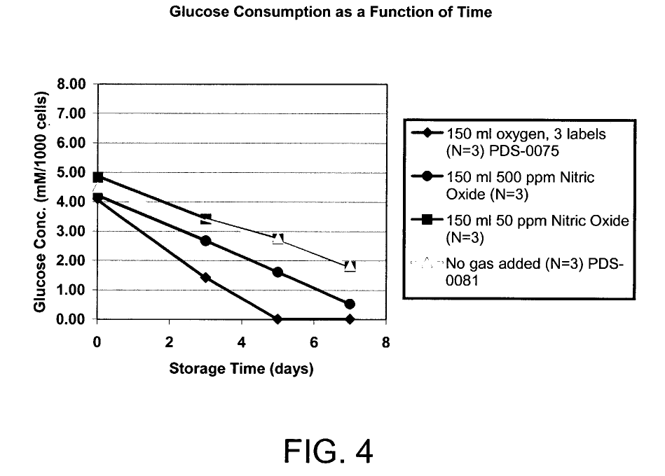

Example 3 was performed using the parameters described in Table 3. Pathogen eradication was performed in a 3L Sengewald bag on 300 ml of plasma (27% carry-over) that was innoculated with bovine viral diarrhea virus (BVDV). The bag also contained 150 ml of 500 ppm nitric oxide gas in a nitrogen balance, delivered through a sterile barrier filter, and 50 μM riboflavin. Mixing was performed before photoradiation at 135 cpm. Visible photoradiation of 419 nm wavelength delivered 10 J/cm² of energy, at about 28° C. Viral kill was monitored before photoradiation and at day 0 immediately after treatment. The results of pathogen inactivation immediately after treatment were compared to other experimental results using an identical visible light process with 100% oxygen and also with no gas added. There was little BVDV inactivation with this nitric oxide treatment, most likely due to the absence of sufficient oxygen in the system. The cell quality was compared to other experimental results using an identical visible light process with 100% oxygen and with no gas added. Cell quality (CQ) results are shown in Table 4, as compared to the oxygen treatment. Cell quality with the nitric oxide treatment was improved over the oxygen treatment and similar to that of the treatment without gas. HSR and GMP-140 were improved using the nitric oxide treatment over treatment without gas. See FIGS. 1-4.

### EXAMPLE 4

Effect of 50 ppm NO on PET Visible Light Process and CQ, using Plasma and BVDV

Example 4 was performed using the parameters described in Table 5. Treatment 4 was identical to Treatment 3, except that 50 ppm nitric oxide gas was used. Cell quality results are shown in Table 6, as compared to the oxygen treatment. The cell quality of the 50 ppm nitric oxide treatment was improved over the 500 ppm nitric oxide treatment in Example 3. See FIGS. 1-4.

### EXAMPLE 5

Effect of 500 ppm NO on PET UV Process, using Plasma and BVDV

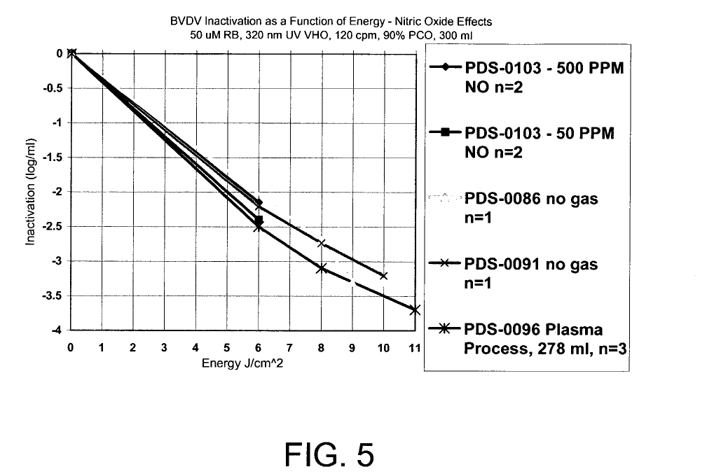

Example 5 was performed using the parameters described in Table 7. Pathogen eradication was performed in a 3L Sengewald bag on 300 ml of plasma (90% carry-over) that was innoculated with bovine viral diarrhea virus (BVDV). The bag also contained 150 ml of 500 ppm nitric oxide gas in a nitrogen balance, delivered through a sterile barrier filter, and 50 μM riboflavin. Mixing was performed before photoradiation at 120 cpm. Ultraviolet photoradiation of 320 nm wavelength delivered 6 J/cm² of energy, at about 30° C. Viral kill was monitored before photoradiation and at day 0 immediately after treatment. The results from this treatment were compared to results from a similar treatment using no nitric oxide gas, 278 ml fluid in a 3L bag, and with T8 bulbs and icecap ballasts. The two lighting configurations provide similar results. See FIG. 5.

### EXAMPLE 6

Effect of 50 ppm NO on PET UV Process, using Plasma and BVDV

Example 6 was performed using the parameters described in Table 8. Treatment 6 was identical to Treatment 5, except that 50 ppm nitric oxide was used. The BVDV inactivation kinetics for these two concentrations of nitric oxide gas appear to be similar to that of no-gas added tests to which they were compared. See FIG. 5.

### EXAMPLE 7

Effect of 500 ppm NO on CQ During PET UV Process, using Platelets

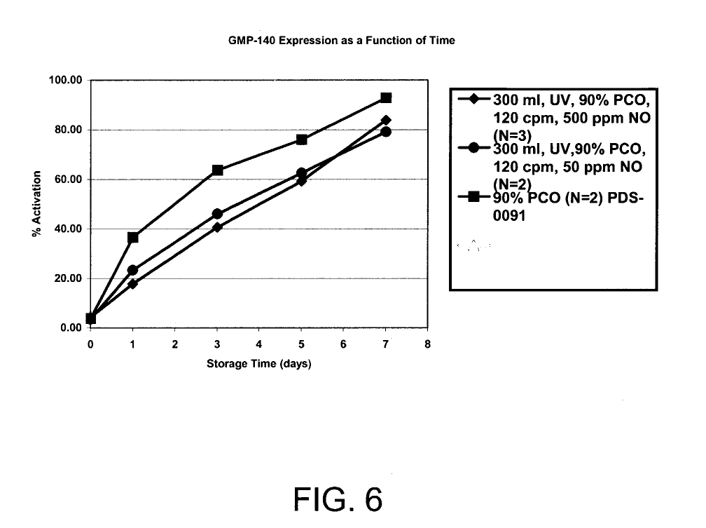

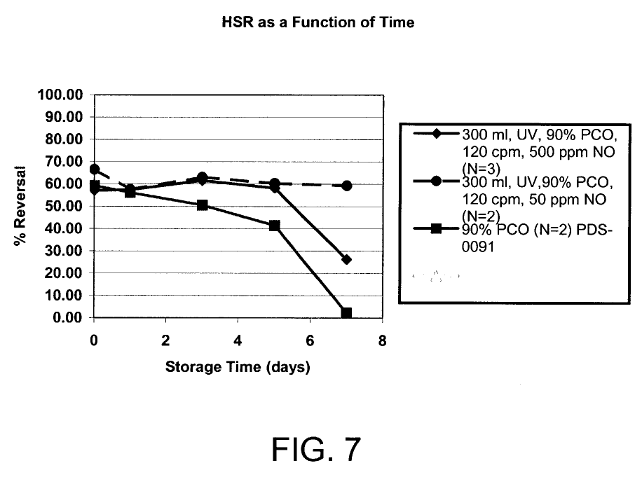

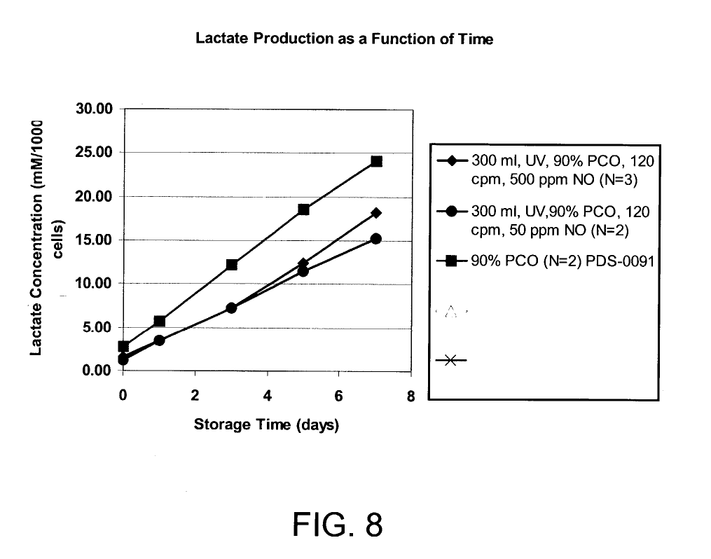

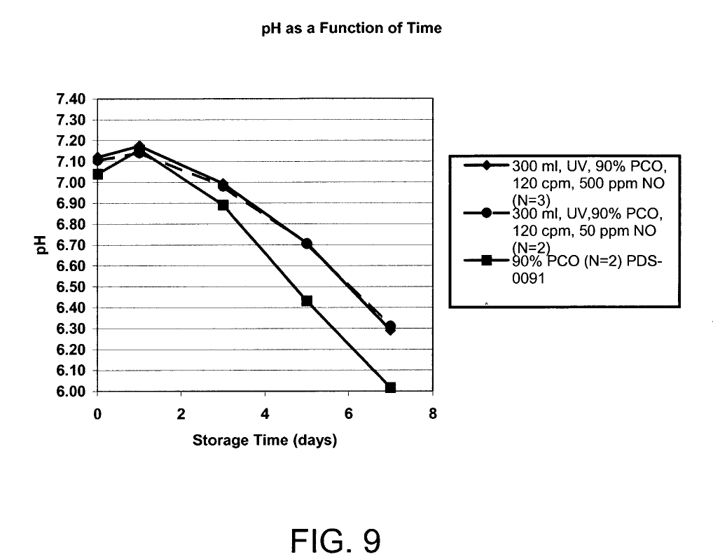

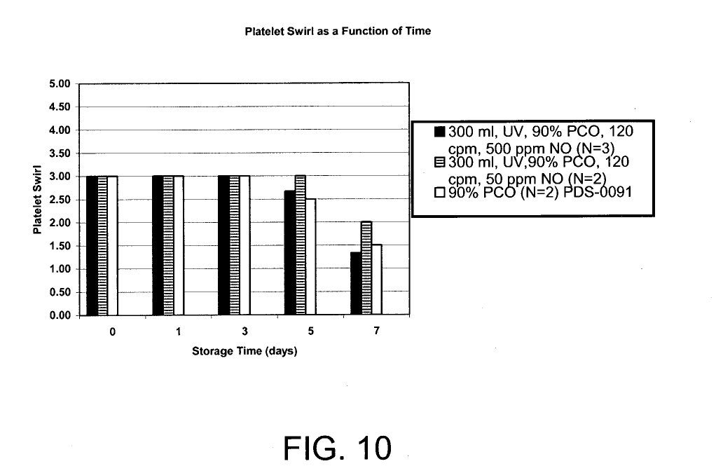

Example 7 was performed using the parameters described in Table 9. Pathogen eradication was performed in a 3L Sengewald bag on 300 ml of platelets that was not innoculated. The bag also contained 150 ml of 500 ppm nitric oxide gas in a nitrogen balance, delivered through a sterile barrier filter, and 50 μM riboflavin. Mixing was performed before photoradiation at 120 cpm. Ultraviolet photoradiation of 320 nm wavelength delivered 6 J/cm² of energy, at about 30° C. Cell quality results are shown in Table 10. The 500 ppm nitric oxide treatment provided improved cell quality over no gas treatments. See FIGS. 6-10.

### EXAMPLE 8

Effect of 50 ppm NO on CQ During PET UV Process, using Platelets

Example 8 was performed using the parameters described in Table 11. Treatment 8 was identical to Treatment 7, except that 50 ppm nitric oxide was used. Cell quality results are shown in Table 12. Treatments with 50 ppm nitric oxide had improved cell quality indicators over the 500 ppm treatment. See FIGS. 6-10.

### EXAMPLE 9

Effect of 25 ppm NO with Oxygen on PET UV Process, using Plasma and E. coli

Example 9 was performed using the parameters described in Table 13. Pathogen eradication was performed in a 3L Sengewald bag on 278 ml of plasma (90% carry-over) that was innoculated with E. coli. The bag also contained 150 ml of 25 ppm nitric oxide gas in an air balance, delivered through a sterile barrier filter, and 50 μM riboflavin. Mixing was performed before photoradiation at 120 cpm. Ultraviolet photoradiation of 320 nm wavelength delivered 8 J/cm² of energy, at about 30° C. Bacteria kill was monitored before photoradiation, at day 0 immediately after treatment at day 1. Positive controls were prepared that underwent every step except for photoradiation. The bacterial titer at one day for the positive controls was compared to the titer for the samples that were photoradiated.

### EXAMPLE 10

Effect of 25 ppm NO on PET UV Process, using Plasma and E. coli

Example 10 was performed using the parameters described in Table 14. Treatment 10 was identical to treatment 9, except that a nitrogen balance was used instead of an air balance. Treatments with an air balance had improved pathogen inactivation over the nitrogen balance treatment.

### EXAMPLE 11

Effect of 25 ppm NO in Oxygen on PET UV Process and CQ, using Plasma and S. epidermidis

Example 11 was performed using the parameters described in Table 15. Treatment 11 was identical to treatment 9, except that S. epidermidis was used as the pathogen.

### EXAMPLE 12

Effect of 25 ppm NO on PET UV Process, using Plasma and E. Coli

Example 12 was performed using the parameters described in Table 16. Treatment 12 was identical to treatment 11, except that a nitrogen balance was used instead of an air balance. Treatments with an air balance had improved pathogen inactivation over the nitrogen balance treatment.

### EXAMPLE 13

Effect of 50 ppm NO on CQ During High-Energy PET UV Process, using Platelets

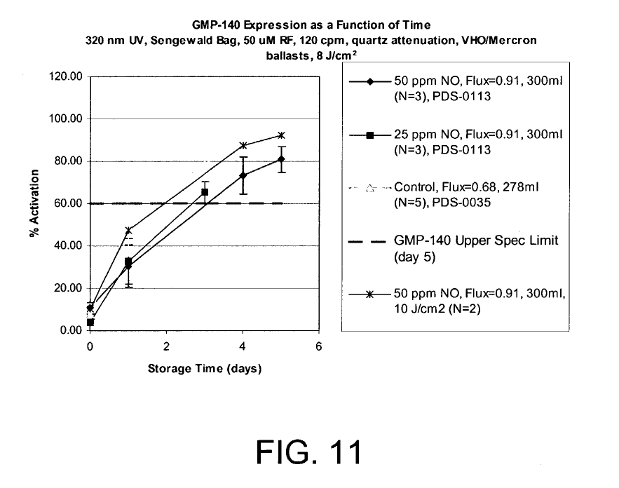

Example 13 was performed using the parameters described in Table 17. Pathogen eradication was performed in a 3L Sengewald bag on 300 ml of platelets that was not innoculated. The bag also contained 150 ml of 50 ppm nitric oxide gas in a nitrogen balance, delivered through a sterile barrier filter, and 50 μM riboflavin. Mixing was performed before photoradiation at 120 cpm. Ultraviolet photoradiation of 320 nm wavelength delivered 8 J/cm² of energy, at about 30° C. Cell quality results are shown in Table 18. Additional cell quality indicators were measured including ATP tests and platelet aggregation. See FIGS. 11 -16.

### EXAMPLE 14

Effect of 50 ppm NO on CQ

During Very High-Energy PET UV Process, Using Platelets

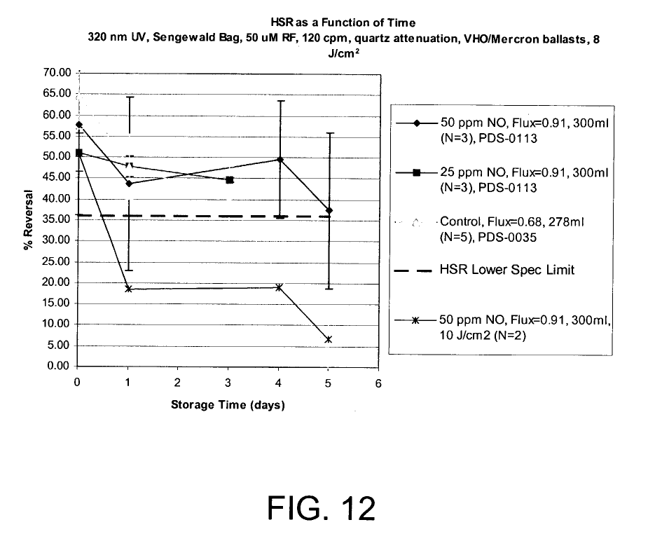

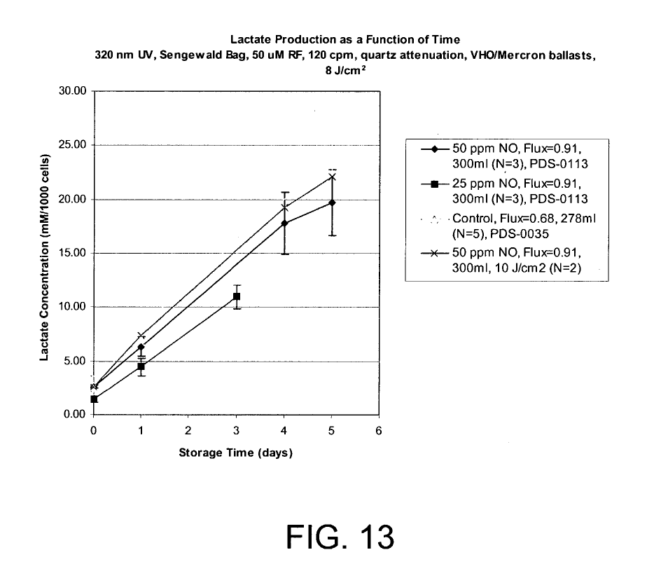

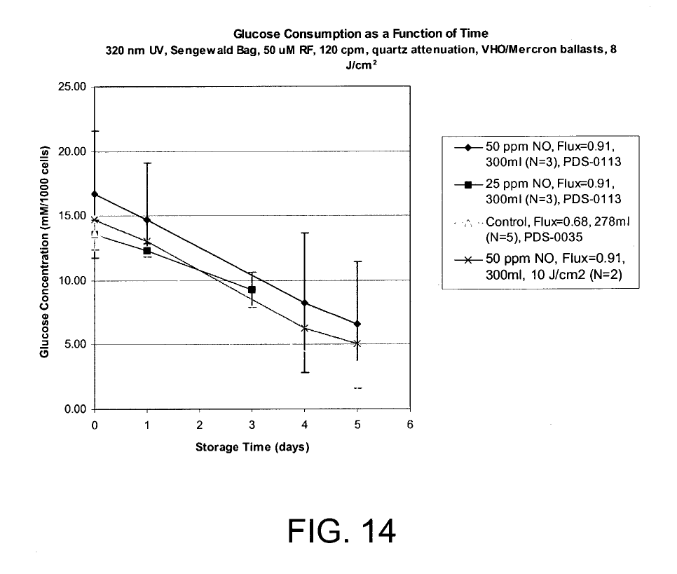

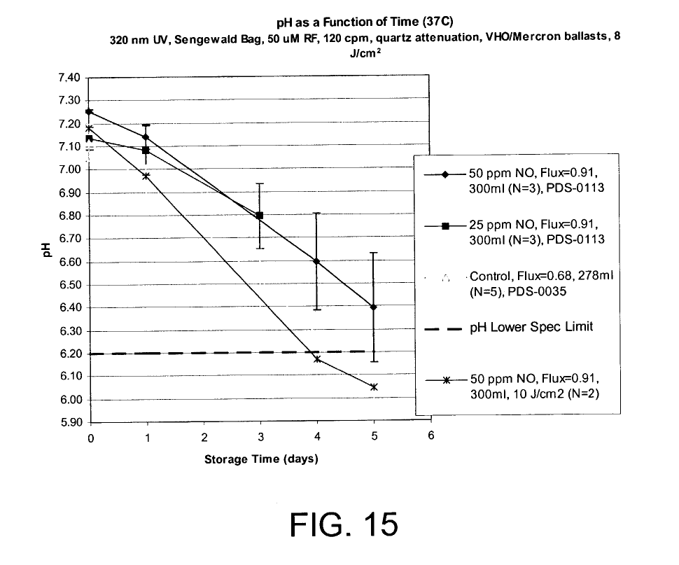

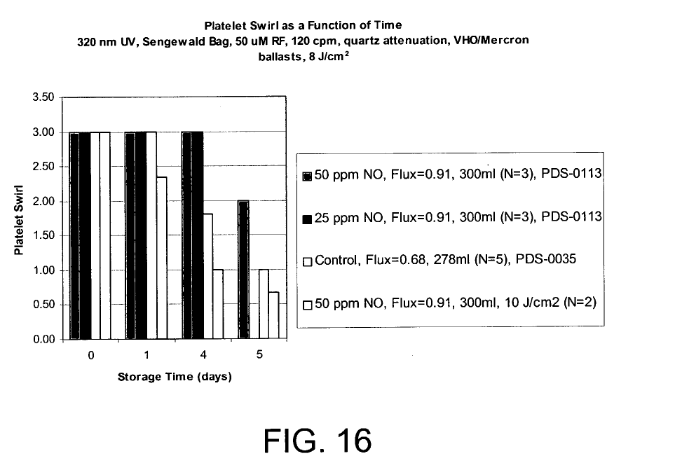

Example 14 was performed using the parameters described in Table 19. Treatment 14 was identical to Treatment 13, except that 10 J/cm² energy was applied. Cell quality results are shown in Table 20. See FIGS. 11-16.

### EXAMPLE 15

Effect of 25 ppm NO on CQ During High-Energy PET UV Process, using Platelets

Example 15 was performed using the parameters described in Table 21. Treatment 15 was identical to Treatment 13, except that 25 ppm nitric oxide was used. Cell quality results are shown in Table 22. Treatments with 25 ppm nitric oxide had improved cell quality indicators over the 50 ppm treatment. See FIGS. 11-16.

### EXAMPLE 16

Effect of 25 ppm NO with 40% Oxygen on PET UV Process, using Plasma and E. coli

Treatment 16 is identical to treatment 9, except that the balance gas is 40% oxygen and 60% nitrogen, equally 20% more oxygen than is found in air. Inactivation and cell quality is improved over Treatment 9.

### EXAMPLE 17

Effect of 25 ppm NO in an Oxygen Balance on Cell Quality after PET UV Process, Using Plasma and E. coli

Treatment 17 is identical to Treatment 16, except that the balance gas is 100% oxygen. Inactivation and cell quality is improved over treatment 16.

### EXAMPLE 18

Effect of NO During Storage on Cell Quality after PET UV Process, using Platelets and E. coli

Treatment 18 is identical to Treatment 15 except that E. coli was utilized as a pathogen and 25 ppm nitric oxide gas was added to the new blood bag after the photoradiated fluid was transferred to the new bag for storage. Cell quality indicators show slight improvement in cell quality during storage with addition of nitric oxide after photoradiation and before storage.

### EXAMPLE 19

Effect of Adding Quencher to Cell Quality after PET UV Process, using Platelets

Treatment 19 is identical to Treatment 15 except that quencher is added to the fluid before photoradiation. Cell quality indicators show improvement using quencher compared to Treatment 15.

### EXAMPLE 20

Effect of NO Gas on Bacterial Growth

E. coli was used to innoculate bacterial growth broth at approximately 1×10⁶ bacteria/ml. Approximately 200 ml was placed in a 300 ml erhlenmeyer flasks. Control flasks were filled with air. Test flasks were filled with 500 ppm nitric oxide in a nitrogen balance. Prepared flasks were incubated with shaking at 37° C. Visual inspection showed large-scale bacteria growth in both flask categories after two and a half days of incubation. It was concluded that nitric oxide by itself did not kill E. coli or inhibit its growth.

### EXAMPLE 21

Effect of NO on PET Visible Process using an Air Balance, Plasma, and BVDV

Treatment 21 is identical to Treatment 4 except that it uses an air balance gas instead of a nitrogen balance gas. Pathogen inactivation is improved over treatment without nitric oxide.

### EXAMPLE 22

Effect of NO on PET Visible Process using a 100% Oxygen Balance, Plasma, and BVDV

Treatment 22 is identical to Treatment 21 except that a 100% oxygen balance gas was used instead of an air balance gas. Pathogen inactivation is improved over treatment 21.

### EXAMPLE 23

Effect of 300 μM N-acetyl-L-cysteine on Cell Quality after PET UV Process

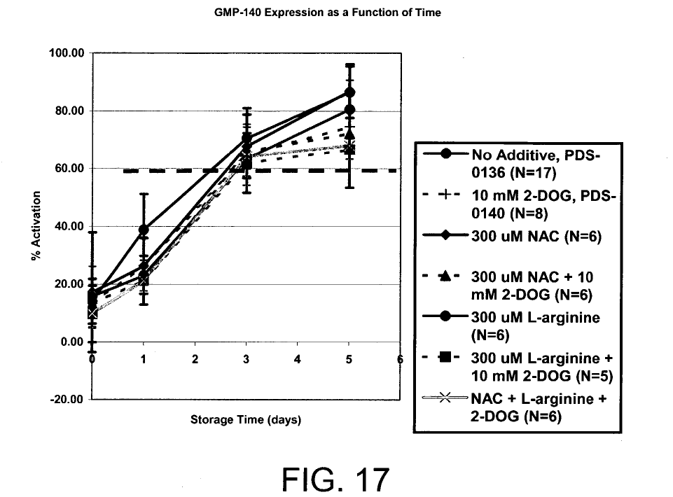

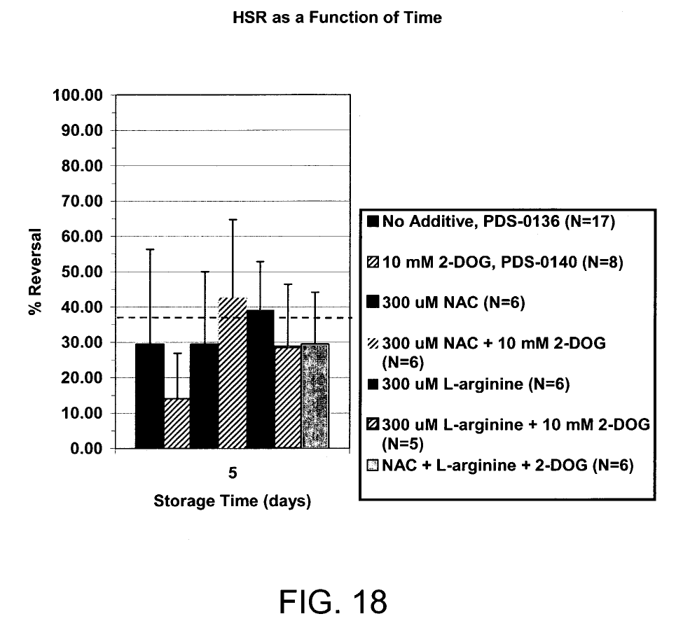

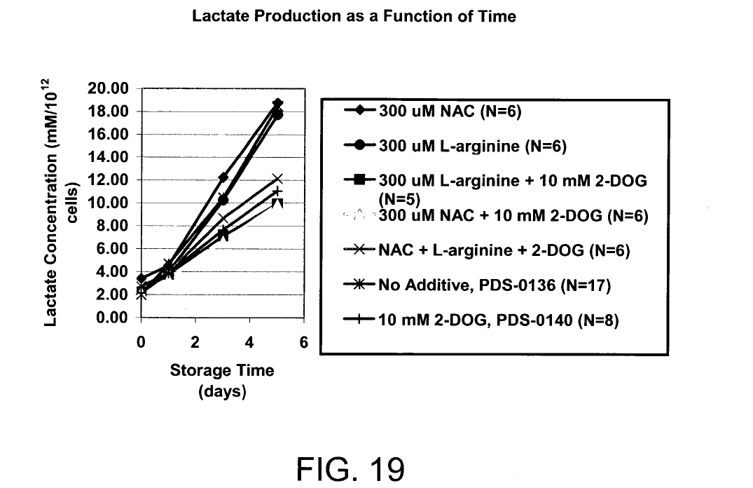

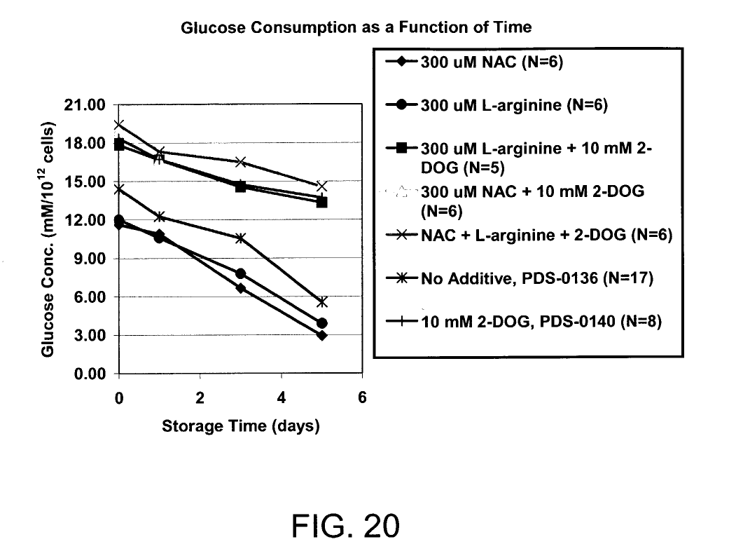

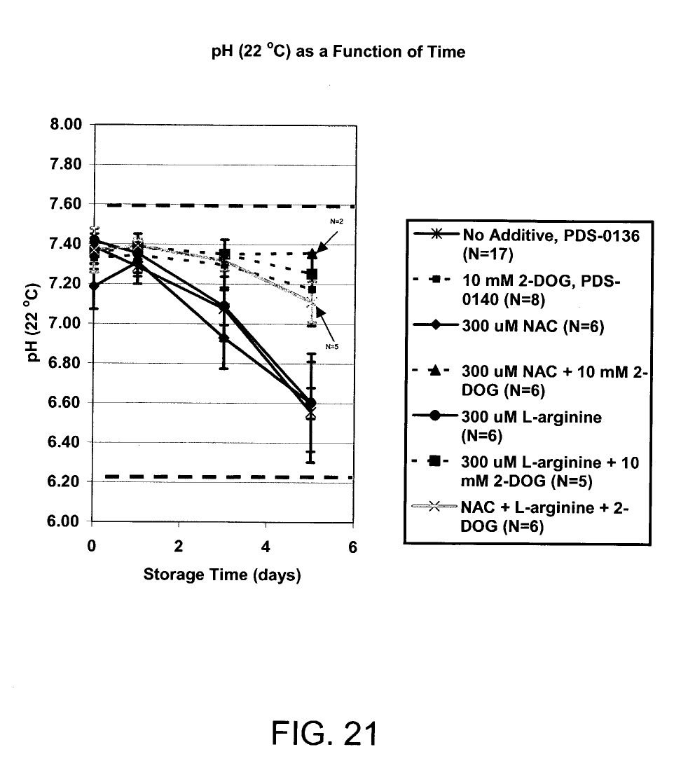

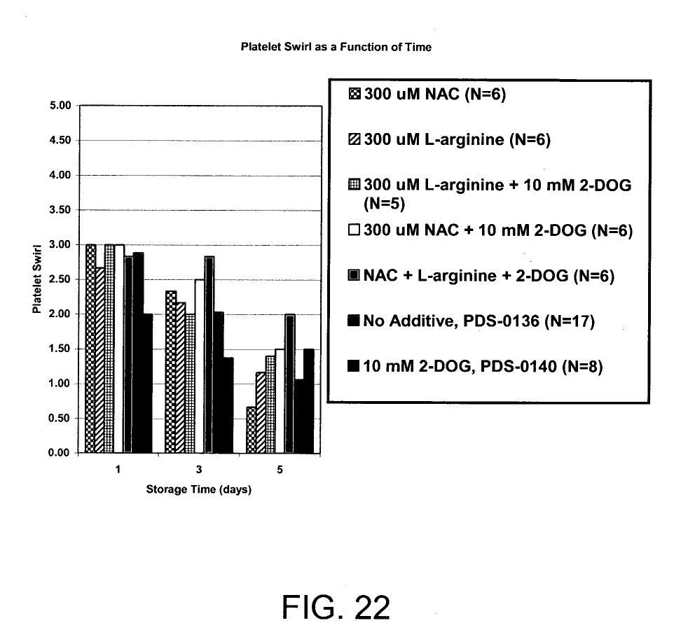

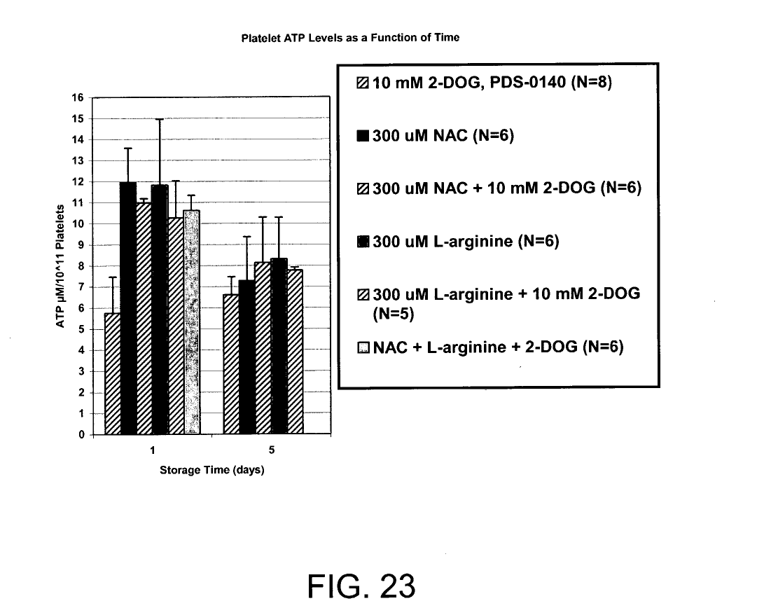

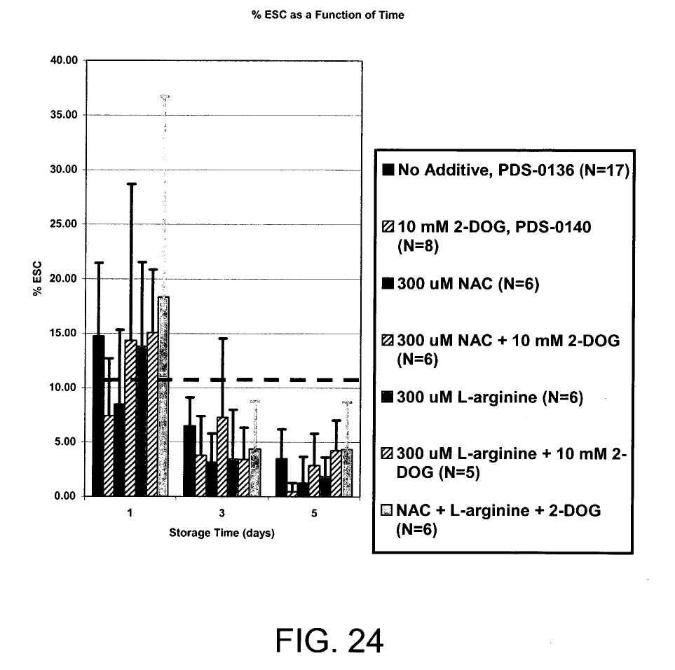

Example 23 was performed using the parameters described in Table 23. Pathogen eradication was performed in a 3L Sengewald bag on 278 ml of fluid comprising platelets. The bag also contained 300 μM N-acetyl-L-cysteine and 50 μM riboflavin. Mixing was performed before photoradiation at 120 cpm. Ultraviolet photoradiation of 320 nm wavelength delivered at total of 7 J/cm² of energy, at about 30° C. Cell quality indicators were measured after pathogen inactivation. The results from this treatment were compared to results from a similar treatment without NO generator. See FIGS. 17-24 and Table 33.

### EXAMPLE 24

Effect of 300 μM L-arginine on Cell Quality after PET UV Process using Platelets

Example 24 was performed using the parameters described in Table 24. Pathogen eradication was performed in a 3L Sengewald bag on 278 ml of fluid comprising platelets. The bag also contained 300 μM L-arginine and 50 μM riboflavin. Mixing was performed before photoradiation at 120 cpm. Ultraviolet photoradiation of 320 nm wavelength delivered at total of 7 J/cm² of energy, at about 30° C. Cell quality indicators were measured after pathogen inactivation. The results from this treatment were compared to results from a similar treatment without NO generator. See FIGS. 17-24 and Table 33.

### EXAMPLE 25

Effect of 300 μM N-acetyl-L-cysteine on Cell Quality after PET UV Process using Platelets

Example 25 was performed using the parameters described in Table 25. Pathogen eradication was performed in a 3L Sengewald bag on 278 ml of fluid comprising platelets. The bag also contained 300 μM N-acetyl-L-cysteine and 50 μM riboflavin. Mixing was performed before photoradiation at 120 cpm. Ultraviolet photoradiation of 320 nm wavelength delivered at total of 7 J/cm² of energy, at about 30° C. Cell quality indicators were measured after pathogen inactivation. The results from this treatment were compared to results from a similar treatment without NO generator. See FIGS. 17-24 and Table 33.

### EXAMPLE 26

Effect of 300 μM L-arginine and 10 mM 2-deoxy-D-glucose on Cell Quality after PET UV Process using Platelets

Example 26 was performed using the parameters described in Table 26. Pathogen eradication was performed in a 3L Sengewald bag on 278 ml of fluid comprising platelets. The bag also contained 300 μM L-arginine, 10 mM 2-deoxy-D-glucose, and 50 μM riboflavin. Mixing was performed before photoradiation at 120 cpm. Ultraviolet photoradiation of 320 nm wavelength delivered at total of 7 J/cm² of energy, at about 30° C. Cell quality indicators were measured after pathogen inactivation. The results from this treatment were compared to results from a similar treatment without NO generator and without glycolysis inhibitor. See FIGS. 17-24 and Table 33.

### EXAMPLE 27

Effect of 300 μM L-arginine and 10 Millimolar 2-deoxy-D-glucose on Cell Quality after PET UV Process using Platelets at a Lower Flux

Example 27 was performed using the parameters described in Table 27. Pathogen eradication was performed in a 3L Sengewald bag on 278 ml of fluid comprising platelets. The bag also contained 300 μM L-arginine, 10 mM 2-deoxy-D-glucose, and 50 μM riboflavin. Mixing was performed before photoradiation at 120 cpm. Ultraviolet photoradiation of 320 nm wavelength delivered at total of 7 J/cm² of energy, at about 30° C., but at only a flux of 0.43 and for 16.3 minutes. Cell quality indicators were measured after pathogen inactivation. The results from this treatment were compared to results from a similar treatment without NO generator and without glycolysis inhibitor.

### EXAMPLE 28

Effect of 300 μM N-acetyl-L-cysteine and 10 mM 2-deoxy-D-glucose on Cell Quality after PET UV Process using Platelets

Example 28 was performed using the parameters described in Table 28. Pathogen eradication was performed in a 3L Sengewald bag on 278 ml of fluid comprising platelets. The bag also contained 300 μM N-acetyl-L-cysteine, 10 mM 2-deoxy-D-glucose, and 50 μM riboflavin. Mixing was performed before photoradiation at 120 cpm. Ultraviolet photoradiation of 320 nm wavelength delivered at total of 7 J/cm² of energy, at about 30° C. Cell quality indicators were measured after pathogen inactivation. The results from this treatment were compared to results from a similar treatment without NO generator and without glycolysis inhibitor. See FIGS. 17-24.

### EXAMPLE 29

Effect of 300 μM L-arginine, 300 μM N-acetyl-L-cysteine, and 10 mM 2-deoxy-D-glucose on Cell Quality after PET UV Process using Platelets

Example 29 was performed using the parameters described in Table 29. Pathogen eradication was performed in a 3L Sengewald bag on 278 ml of fluid comprising platelets. The bag also contained 300 μM N-acetyl-L-cysteine, 300 μM L-arginine, 10 mM 2-deoxy-D-glucose, and 50 μM riboflavin. Mixing was performed before photoradiation at 120 cpm. Ultraviolet photoradiation of 320 nm wavelength delivered at total of 7 J/cm² of energy, at about 30° C. Cell quality indicators were measured after pathogen inactivation. The results from this treatment were compared to results from a similar treatment without NO generator and without glycolysis inhibitor. See FIGS. 17-24 and Table 33.

### EXAMPLE 30

Effect of 300 μM L-arginine on BVDV Inactivation using PET UV Process with Increasing Energy Using Plasma

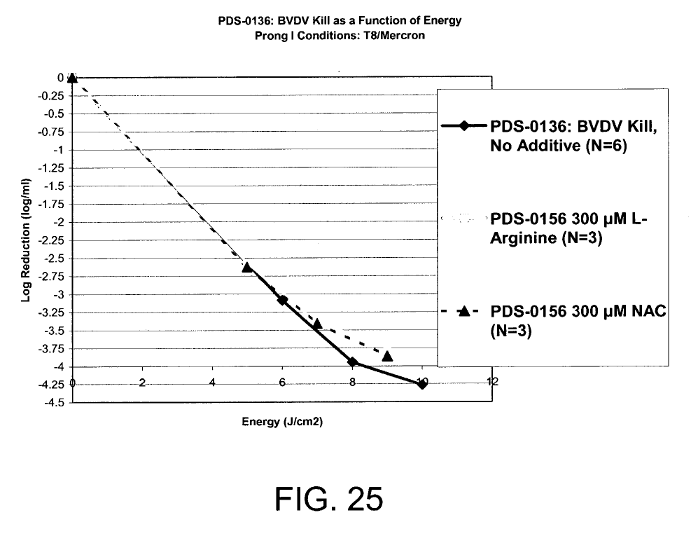

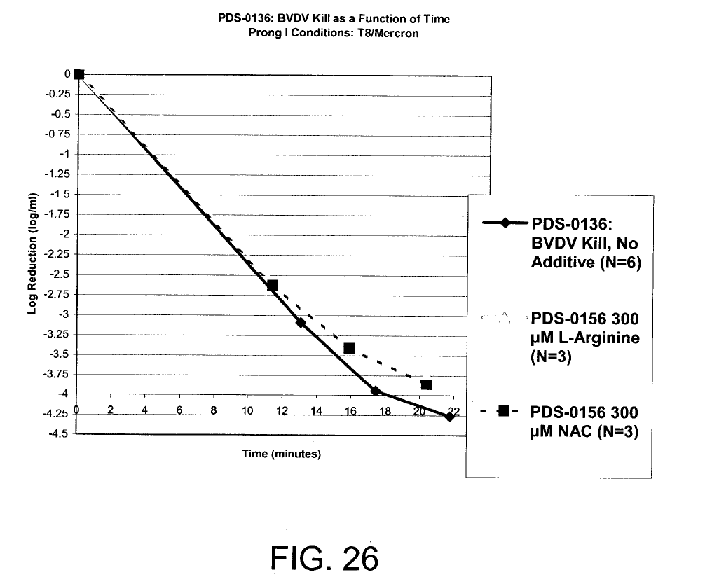

Example 30 was performed using the parameters described in Table 30. Pathogen eradication was performed in a 3L Sengewald bag on 278 ml of plasma (85% carry-over) that was innoculated with bovine viral diarrhea virus (BVDV). The bag also contained 300 μM L-arginine and 50 μM riboflavin. Mixing was performed before photoradiation at 120 cpm. Ultraviolet photoradiation of 320 nm wavelength delivered at total of 9 J/cm² of energy, at about 30° C. Viral kill was monitored before photoradiation, and at delivery of 5 J/cm², 7 J/cm², and 9 J/cm². The results from this treatment were compared to results from a similar treatment without NO generator. L-arginine did not interfere with pathogen inactivation. See FIGS. 25 and 26 and Table 33.

### EXAMPLE 31

Effect of 300 μM N-acetyl-L-cysteine on BVDV Inactivation using PET UV Process with Increasing Energy using Plasma

Example 31 was performed using the parameters described in Table 31. Pathogen eradication was performed in a 3L Sengewald bag on 278 ml of plasma (85% carry-over) that was innoculated with bovine viral diarrhea virus (BVDV). The bag also contained 300 μM N-acetyl-L-cysteine and 50 μM riboflavin. Mixing was performed before photoradiation at 120 cpm. Ultraviolet photoradiation of 320 nm wavelength delivered at total of 9 J/cm² of energy, at about 30° C. Viral kill was monitored before photoradiation, and at delivery of 5 J/cm², 7 J/cm², and 9 J/cm². The results from this treatment were compared to results from a similar treatment without NO generator. N-acetyl-cysteine did not interfere with pathogen inactivation. See FIGS. 25 and 26 and Table 33.

### EXAMPLE 32

Effect of 300 μM N-acetyl-L-cysteine and 10 mM 2-deoxy-D-glucose on BVDV Inactivation using PET UV Process with Increasing Energy using Plasma

Example 32 was performed using the parameters described in Table 32. Pathogen eradication was performed in a 3L Sengewald bag on 278 ml of plasma (85% carry-over) that was innoculated with bovine viral diarrhea virus (BVDV). The bag also contained 300 μM N-acetyl-L-cysteine, 10 mM 2-deoxy-D-glucose, and 50 μM riboflavin. Mixing was performed before photoradiation at 120 cpm. Ultraviolet photoradiation of 320 nm wavelength delivered at total of 9 J/cm² of energy, at about 30° C. Viral kill was monitored before photoradiation, and at delivery of 5 J/cm², 7 J/cm², and 9 J/cm². The results from this treatment were compared to results from a similar treatment without NO generators and without glycolysis inhibitor.

### EXAMPLE 33

Effect of 300 μM N-acetyl-L-cysteine, 300 μM L-Arginine, 10 mM 2-deoxy-D-glucose, and 25 ppm Nitric Oxide Gas on Cell Quality using PET UV Process using Platelets

Treatment 33 was performed identically as Treatment 29 except that 25 ppm nitric oxide gas was added before photoradiation. Cell quality indicators were improved over those in Treatments 15 and 29.

### EXAMPLE 34

Effect of 300 μM N-acetyl-L-cysteine, 300 μM L-Arginine, 10 mM 2-deoxy-D-glucose, 25 ppm Nitric Oxide Gas and Quencher on Cell Quality using PET UV Process using Platelets

Treatment 34 was performed identically as Treatment 33 except that quencher was added before photoradiation. Cell quality indicators were improved over those in Treatments 19, 29, and 33.

### EXAMPLE 35

Effect of 300 μM N-acetyl-L-cysteine, 300 μM L-Arginine 10 mM 2-deoxy-D-glucose, 25 ppm Nitric Oxide Gas, and/or Quencher on Cell Quality using PET Visible Process using Platelets

Treatment 35 photoradiation was performed identically as Treatment 3 except that various combinations of 300 μM N-acetyl-L-cysteine, 300 μM L-Arginine, 10 mM 2-deoxy-D-glucose, 25 ppm Nitric Oxide Gas, and/or quencher were added before photoradiation. Cell quality indicators were improved over a similar visible light pathogen inactivation process without added nitric oxide, glycolysis inhibitor, or quencher.

### EXAMPLE 36

Effect of 300 μM N-acetyl-L-cysteine, 300 μM L-Arginine and 10 mM 2-deoxy-D-glucose on E. coli Inactivation using PET UV Process with Increasing Energy Using Plasma

Treatment 36 was identical to Treatment 32 except that E. coli was used as a pathogen instead of BVDV. Pathogen inactivation was successful.

TABLE 1

| | Example | | |
|---|---|---|---|
| | 1 | 1 | 1 |
| bag size | 3 L | 3 L | 3 L |
| total fluid volume (ml) | 300 | 300 | 300 |
| aspect ratio | .43 | .43 | .43 |
| blood component | plasma | plasma | plasma |
| | (90% CO) | (90% CO) | (90% CO) |
| Riboflavin | 50 | 50 | 50 |
| (micromolar) | | | |
| gas volume | 150 ml | 150 ml | 150 ml |
| nitric oxide gas | 500 ppm | 500 ppm | 500 ppm |
| Nitrogen | balance | balance | balance |
| air (oxygen) | NA | NA | NA |
| Photoradiation | UV | UV | UV |
| Wavelength | 320 nm | 320 nm | 320 nm |
| energy (J/cm²) | 6 | 6 | 6 |
| Mylar | none | none | none |
| Exposure time | 6.6 | 6.6 | 6.6 |
| (minutes) | | | |
| light configuration | VHO/Mercron | VHO/Mercron | VHO/Mercron |
| mixing speed (cpm) | 120 | 120 | 120 |
| pathogen | E. coli | E. coli | E. coli |
| pathogen titer | 5.86 | 5.79 | 5.82 |
| (log, T0) | | | |
| pathogen titer | 2.53 | 2.11 | 2.00 |
| (log, T1) | | | |
| pathogen kill | −3.33 | −3.68 | −3.82 |
| (log, T0 − T1) | | | |
| pathogen kill (%) | 99.95 | 99.98 | 99.98 |
| temperature (° C.) | ˜30 | ˜30 | ˜30 |

TABLE 2

| | Example | | |
|---|---|---|---|
| | 2 | 2 | 2 |
| bag size | 3 L | 3 L | 3 L |
| total fluid volume (ml) | 300 | 300 | 300 |
| aspect ratio | .43 | .43 | .43 |
| blood component | plasma (90% CO) | plasma (90% CO) | plasma (90% CO) |
| riboflavin (micromolar) | 50 | 50 | 50 |
| gas volume | 150 ml | 150 ml | 150 ml |
| nitric oxide gas | 500 ppm | 500 ppm | 500 ppm |
| nitrogen | balance | balance | balance |
| air (oxygen) | NA | NA | NA |
| photoradiation | UV | UV | UV |
| wavelength | 320 nm | 320 nm | 320 nm |
| energy (J/cm²) | 6 | 6 | 6 |
| mylar | none | none | none |
| exposure time (minutes) | 6.6 | 6.6 | 6.6 |
| light configuration | VHO/Mercron | VHO/Mercron | VHO/Mercron |
| mixing speed (cpm) | 120 | 120 | 120 |
| pathogen | S. epidermidis | S. epidermidis | S. epidermidis |
| pathogen titer (log, T0) | 5.54 | 5.82 | 5.72 |
| pathogen titer (log, T1) | 1.30 | 1.30 | 1.00 |
| pathogen kill (log, T1 − T0) | −4.24 | −4.52 | −4.72 |
| pathogen kill (%) | 99.99995 | 99.99997 | 99.99998 |
| temperature (° C.) | ˜30 | ˜30 | ˜30 |

TABLE 3

| | Example | | |
|---|---|---|---|
| | 3 | 3 | 3 |
| bag size | 3 L | 3 L | 3 L |
| total fluid volume (ml) | 300 | 300 | 300 |
| aspect ratio | .43 | .43 | .43 |
| blood component | plasma (27% CO) | plasma (27% CO) | plasma (27% CO) |
| riboflavin (micromolar) | 50 | 50 | 50 |
| gas volume | 150 ml | 150 ml | 150 ml |
| nitric oxide gas | 500 ppm | 500 ppm | 500 ppm |
| nitrogen | balance | balance | Balance |
| air (oxygen) | NA | NA | NA |
| photoradiation | visible | visible | Visible |
| wavelength | 419 nm | 419 nm | 419 nm |
| energy (J/cm²) | 10 | 10 | 10 |
| mylar | 2 sheets | 2 sheets | 2 sheets |
| exposure time (minutes) | 7.1 | 7.1 | 7.1 |
| light configuration | VHO/Mercron | VHO/Mercron | VHO/Mercron |
| mixing speed (cpm) | 135 | 135 | 135 |
| pathogen | BVDV | BVDV | BVDV |
| pathogen titer (log, T0) | 6.2 | 6.4 | 6.1 |
| pathogen titer (log, T1) | 6.2 | 5.7 | 6.5. |
| pathogen kill (log, T1 − T0) | 0.0 | −1.2 | 0.4 |
| pathogen kill (%) | 0 | 93.7 | NA |
| temperature (° C.) | ˜28 | ˜28 | ˜28 |

TABLE 4

| Example 3 | | | |
|---|---|---|---|
| cell quality measurement | Day 3 | Day 5 | Day 7 |
| activation (GMP-140) decrease | 66 | 63 | 68 |
| HSR increase (% reversal) | | 95 | 83 |
| glucose consumption rate decrease % | 88 | NA | NA |
| (mMol/1000 cells) | | | |

TABLE 5

| | Example | | |
|---|---|---|---|
| | 4 | 4 | 4 |
| bag size | 3 L | 3 L | 3 L |
| total fluid volume (ml) | 300 | 300 | 300 |
| aspect ratio | .43 | .43 | .43 |
| blood component | plasma (27% CO) | plasma (27% CO) | plasma (27% CO) |
| riboflavin (micromolar) | 50 | 50 | 50 |
| gas volume | 150 ml | 150 ml | 150 ml |
| nitric oxide gas | 50 ppm | 50 ppm | 50 ppm |
| Nitrogen | balance | balance | Balance |
| air (oxygen) | NA | NA | NA |
| Photoradiation | visible | visible | Visible |
| Wavelength | 419 nm | 419 nm | 419 nm |
| energy (J/cm²) | 10 | 10 | 10 |
| Mylar | 2 sheets | 2 sheets | 2 sheets |
| exposure time (minutes) | 7.1 | 7.1 | 7.1 |
| light configuration | VHO/Mercron | VHO/Mercron | VHO/Mercron |
| mixing speed (cpm) | 135 | 135 | 135 |
| Pathogen | BVDV | BVDV | BVDV |
| pathogen titer (log, T0) | 6.4 | 6.6 | 6.7 |
| pathogen titer (log, T1) | 6.2 | 6.4 | 6.5 |
| pathogen kill (log, T1 − T0) | −0.2 | −0.2 | −0.2 |
| pathogen kill (%) | 36.9 | 36.9 | 36.9 |
| temperature (° C.) | ˜28 | ˜28 | ˜28 |

TABLE 6

| Example 4 | | | |
|---|---|---|---|
| cell quality measurement | Day 3 | Day 5 | Day 7 |
| Activation (GMP-140) decrease | 66 | 64 | 65 |
| HSR increase (% reversal) | | 75 | 85 |
| Glucose consumption rate decrease % | 140 | NA | NA |
| (mMol/1000 cells) | | | |

TABLE 7

| | Example | |
|---|---|---|
| | 5 | 5 |
| bag size | 3 L | 3 L |
| total fluid volume (ml) | 300 | 300 |
| aspect ratio | .43 | .43 |
| blood component | plasma (90% CO) | plasma (90% CO) |
| riboflavin (micromolar) | 50 | 50 |
| gas volume | 150 ml | 150 ml |
| nitric oxide | 500 ppm | 500 ppm |
| nitrogen | balance | balance |
| air (oxygen) | NA | NA |
| photoradiation | UV | UV |
| wavelength | 320 nm | 320 nm |
| energy (J/cm²) | 6 | 6 |
| mylar | none | none |
| exposure time (minutes) | 6.6 | 6.6 |
| light configuration | VHO/Mercron | VHO/Mercron |
| mixing speed (cpm) | 120 | 120 |
| pathogen | BVDV | BVDV |
| pathogen titer (log, T0) | 6.5 | 6.4 |
| pathogen titer (log, T1) | 4.4 | 4.2 |
| pathogen kill (log, T1 − T0) | −2.1 | −2.2 |
| pathogen kill (%) | 99.2 | 99.37 |
| temperature (° C.) | ˜30 | ˜30 |

TABLE 8

| | Example | |
|---|---|---|
| | 6 | 6 |
| bag size | 3 L | 3 L |
| total fluid volume (ml) | 300 | 300 |
| aspect ratio | .43 | .43 |
| blood component | plasma (90% CO) | plasma (90% CO) |
| riboflavin (micromolar) | 50 | 50 |
| gas volume | 150 ml | 150 ml |
| nitric oxide gas | 50 ppm | 50 ppm |
| nitrogen | balance | Balance |
| air (oxygen and nitrogen) | NA | NA |
| photoradiation | UV | UV |
| wavelength | 320 nm | 320 nm |
| energy (J/cm²) | 6 | 6 |
| mylar | none | None |
| exposure time (minutes) | 6.6 | 6.6 |
| light configuration | VHO/Mercron | VHO/Mercron |
| mixing speed (cpm) | 120 | 120 |
| pathogen | BVDV | BVDV |
| pathogen titer (log, T0) | 6.2 | 6.6 |
| pathogen titer (log, T1) | 4.0 | 4.0 |
| pathogen kill (log, T0 − T1) | −2.2 | −2.6 |
| pathogen kill (%) | 99.37 | 99.75 |
| temperature (° C.) | ˜30 | ˜30 |

TABLE 9

| | Example | | |
|---|---|---|---|
| | 7 | 7 | 7 |
| bag size | 3 L | 3 L | 3 L |
| total fluid volume (ml) | 300 | 300 | 300 |
| aspect ratio | .43 | .43 | .43 |
| blood component | platelets (1.3 × 10⁶/μl) | platelets (0.8 × 10⁶/μl) | platelets (1.4 × 10⁶/μl) |
| riboflavin (micromolar) | 50 | 50 | 50 |
| gas volume | 150 ml | 150 ml | 150 ml |
| nitric oxide gas | 500 ppm | 500 ppm | 500 ppm |
| nitrogen | balance | balance | Balance |
| air (oxygen) | NA | NA | NA |
| photoradiation | UV | UV | UV |
| wavelength | 320 nm | 320 nm | 320 nm |
| energy (J/cm²) | 6 | 6 | 6 |
| mylar | none | none | None |
| exposure time (minutes) | 6.6 | 6.6 | 6.6 |
| light configuration | VHO/Mercron | VHO/Mercron | VHO/Mercron |
| mixing speed (cpm) | 120 | 120 | 120 |
| pathogen | NA | NA | NA |
| temperature (° C.) | ˜30 | ˜30 | ˜30 |

TABLE 10

| Example 7 | | | |
|---|---|---|---|
| cell quality measurement | Day 3 | Day 5 | Day 7 |
| activation (GMP-140) decrease | 36.3 | 22.2 | 9.66 |
| HSR increase (% reversal) | 23.9 | 41.3 | 1120 |
| lactate production rate decrease | 29.6 (40.6) | 8.52 (32.3) | NA (24.7) |
| % (production decrease %) | | | |
| pH rate increase % (pH increase) | 37 (.1) | 19.5 (.28) | NA (.27) |
| platelet swirl increase | NA | 6.8 | NA |

TABLE 11

| | Example | | |
|---|---|---|---|
| | 8 | 8 | 8 |
| bag size | 3 L | 3 L | 3 L |
| total fluid volume (ml) | 300 | 300 | 300 |
| aspect ratio | .43 | .43 | .43 |
| blood component | platelets (1.6 × 10⁶/μl) | platelets (1.3 × 10⁶/μl) | platelets (1.4 × 10⁶/μl) |
| riboflavin (micromolar) | 50 | 50 | 50 |
| gas volume | 150 ml | 150 ml | 150 ml |
| nitric oxide gas | 50 ppm | 50 ppm | 50 ppm |
| nitrogen | balance | balance | Balance |
| air (oxygen and nitrogen) | NA | NA | NA |
| photoradiation | UV | UV | UV |
| wavelength | 320 nm | 320 nm | 320 nm |
| energy (J/cm²) | 6 | 6 | 6 |
| mylar | none | none | None |
| exposure time (minutes) | 6.6 | 6.6 | 6.6 |
| light configuration | VHO/Mercron | VHO/Mercron | VHO/Mercron |
| mixing speed (cpm) | 120 | 120 | 120 |
| temperature (° C.) | ˜30 | ˜30 | ˜30 |

TABLE 12

| Example 8 | | | |
|---|---|---|---|
| cell quality measurement | Day 3 | Day 5 | Day 7 |
| activation (GMP-140) decrease | 27.7 | 17.8 | 14.7 |
| HSR increase (% reversal) | 25 | 45.6 | 2653 |
| lactate (mMol/1000 cells) | 36.8 (40.6) | 33.1 (38) | 33.1 (36.9) |
| production rate decrease | | | |
| % (production decrease %) | | | |
| pH rate increase % (pH unit | 41.1 (.09) | 22.3 (.28) | 2.4 (.29) |
| increase) | | | |
| platelet swirl increase (%) | NA | 20 | 33.3 |

TABLE 13

| | Example | | |
|---|---|---|---|
| | 9 | 9 | 9 |
| bag size | 3 L | 3 L | 3 L |
| total fluid volume (ml) | 278 | 278 | 278 |
| aspect ratio | .40 | .40 | .40 |
| blood component | plasma (90% CO) | plasma (90% CO) | plasma (90% CO) |
| riboflavin (micromolar) | 50 | 50 | 50 |
| gas volume | 150 ml | 150 ml | 150 ml |
| nitric oxide gas | 25 ppm | 25 ppm | 25 ppm |
| nitrogen | NA | NA | NA |
| air (oxygen) | balance | balance | Balance |
| photoradiation | UV | UV | UV |
| wavelength | 320 nm | 320 nm | 320 nm |
| energy (J/cm²) | 8 | 8 | 8 |
| mylar | none | none | None |
| exposure time (minutes) | 9.3 | 9.3 | 9.3 |
| light configuration | VHO/Mercron | VHO/Mercron | VHO/Mercron |
| mixing speed (cpm) | 120 | 120 | 120 |
| pathogen | E. coli | E. coli | E. coli |
| pathogen titer (log, positive | 6.38 | 6.26 | 6.36 |
| control) | | | |
| pathogen titer (log, T1) | 2.61 | 1.3 | 3.36 |
| pathogen | −3.77 | −4.96 | −3.0 |
| kill (log, positive control-T1) | | | |
| pathogen kill (%) | 99.98 | 99.999 | 99.9 |
| temperature (° C.) | ˜31 | ˜31 | ˜31 |

TABLE 14

| | Example | | |
|---|---|---|---|
| | 10 | 10 | 10 |
| bag size | 3 L | 3 L | 3 L |
| total fluid volume (ml) | 278 | 278 | 278 |
| aspect ratio | .40 | .40 | .40 |
| blood component | plasma (90% CO) | plasma (90% CO) | plasma (90% CO) |
| riboflavin (micromolar) | 50 | 50 | 50 |
| gas volume | 150 ml | 150 ml | 150 ml |
| nitric oxide gas | 25 ppm | 25 ppm | 25 ppm |
| nitrogen | balance | balance | Balance |
| air (oxygen and nitrogen) | NA | NA | NA |
| photoradiation | UV | UV | UV |
| wavelength | 320 nm | 320 nm | 320 nm |
| energy (J/cm²) | 8 | 8 | 8 |
| mylar | none | none | None |
| exposure time (minutes) | 9.3 | 9.3 | 9.3 |
| light configuration | VHO/Mercron | VHO/Mercron | VHO/Mercron |
| mixing speed (cpm) | 120 | 120 | 120 |
| pathogen | E. coli | E. coli | E. coli |
| pathogen titer (log, positive | 6.4 | 6.27 | 6.39 |
| control) | | | |
| pathogen titer (log, T1) | 3.6 | 2.28 | 2.00 |
| pathogen | 2.8 | 3.99 | 4.39 |
| kill (log, positive control-T1) | | | |
| pathogen kill (%) | 99.84 | 99.99 | 99.996 |
| temperature (° C.) | ˜31 | ˜31 | ˜31 |

TABLE 15

| | Example | | |
|---|---|---|---|
| | 11 | 11 | 11 |
| bag size | 3 L | 3 L | 3 L |
| total fluid volume (ml) | 278 | 278 | 278 |
| aspect ratio | .40 | .40 | .40 |
| blood component | plasma | plasma | plasma |
| | (90% CO) | (90% CO) | (90% CO) |
| riboflavin | 50 | 50 | 50 |
| (micromolar) | | | |
| gas volume | 150 ml | 150 ml | 150 ml |
| nitric oxide gas | 25 ppm | 25 ppm | 25 ppm |
| nitrogen | NA | NA | NA |
| air (oxygen) | balance | balance | Balance |
| photoradiation | UV | UV | UV |
| wavelength | 320 nm | 320 nm | 320 nm |
| energy (J/cm²) | 8 | 8 | 8 |
| mylar | none | none | None |
| exposure time | 9.3 | 9.3 | 9.3 |
| (minutes) | | | |
| light configuration | VHO/Mercron | VHO/Mercron | VHO/Mercron |
| mixing speed (cpm) | 120 | 120 | 120 |
| pathogen | S. epidermidis | S. epidermidis | S. epidermidis |
| temperature (° C.) | ˜31 | ˜31 | ˜31 |

TABLE 16

| | Example | | |
|---|---|---|---|
| | 12 | 12 | 12 |
| bag size | 3 L | 3 L | 3 L |
| total fluid volume (ml) | 278 | 278 | 278 |
| aspect ratio | .40 | .40 | .40 |
| blood component | plasma | plasma | plasma |
| | (90% CO) | (90% CO) | (90% CO) |
| riboflavin | 50 | 50 | 50 |
| (micromolar) | | | |
| gas volume | 150 ml | 150 ml | 150 ml |
| nitric oxide gas | 25 ppm | 25 ppm | 25 ppm |
| nitrogen | balance | balance | balance |
| air | NA | NA | NA |
| (oxygen and nitrogen) | | | |
| photoradiation | UV | UV | UV |
| wavelength | 320 nm | 320 nm | 320 nm |
| energy | 8 | 8 | 8 |
| (J/cm²) | | | |
| mylar | none | none | none |
| exposure time | 9.3 | 9.3 | 9.3 |
| (minutes) | | | |
| light configuration | VHO/Mercron | VHO/Mercron | VHO/Mercron |
| mixing speed (cpm) | 120 | 120 | 120 |
| pathogen | S. epidermidis | S. epidermidis | S. epidermidis |
| temperature (° C.) | ˜31 | ˜31 | ˜31 |

TABLE 17

| | Example | | |
|---|---|---|---|
| | 13 | 13 | 13 |
| bag size | 3 L | 3 L | 3 L |
| total fluid volume (ml) | 300 | 300 | 300 |
| aspect ratio | .43 | .43 | .43 |
| blood component | platelets (1.4 × 10⁶/μl) | platelets (1.2 × 10⁶/μl) | platelets (1.1 × 10⁶/μl) |
| riboflavin (micromolar) | 50 | 50 | 50 |
| gas volume | 150 ml | 150 ml | 150 ml |
| nitric oxide gas | 50 ppm | 50 ppm | 50 ppm |
| nitrogen | balance | balance | balance |
| air (oxygen) | NA | NA | NA |
| photoradiation | UV | UV | UV |
| wavelength | 320 nm | 320 nm | 320 nm |
| energy (J/cm²) | 8 | 8 | 8 |
| mylar | none | none | none |
| flux | .91 | .91 | .91 |
| exposure time (minutes) | 8.8 | 8.8 | 8.8 |
| light configuration | VHO/Mercron | VHO/Mercron | VHO/Mercron |
| mixing speed (cpm) | 120 | 120 | 120 |
| temperature (° C.) | ˜30 | ˜30 | ˜30 |

TABLE 18

| Example 13 | | | |
|---|---|---|---|
| cell quality measurement | Day 3 | Day 4 | Day 5 |
| activation (GMP-140) | 25 | 16 | 15.2 |
| decrease | | | |
| HSR increase (% reversal) | 6.6 | 120 | 800 |
| lactate (mMol/1000 cells) | 3.2 (16.9) | 17.7 (11.3) | 48.3 (17.2) |
| production rate decrease % | | | |
| (production decrease %) | | | |
| glucose (mMol/1000 cells) | 10.8 | 2.6 | 8.0 |
| consumption rate decrease % | | | |
| pH rate increase % (pH unit | 18.5 (.26) | 17.0 (.32) | 11.1 (.34) |
| increase) | | | |
| platelet swirl increase (%) | 67 (Day 4) | 100 | |

TABLE 19

| | Example | | |
|---|---|---|---|
| | 14 | 14 | 14 |
| bag size | 3 L | 3 L | 3 L |
| total fluid volume (ml) | 300 | 300 | 300 |
| aspect ratio | .43 | .43 | .43 |
| blood component | platelets (1.4 × 10⁶/μl) | platelets (1.4 × 10⁶/μl) | platelets (0.9 × 10⁶/μl) |
| riboflavin (micromolar) | 50 | 50 | 50 |
| gas volume | 150 ml | 150 ml | 150 ml |
| nitric oxide gas | 50 ppm | 50 ppm | 50 ppm |
| nitrogen | balance | balance | balance |
| air (oxygen) | NA | NA | NA |
| photoradiation | UV | UV | UV |
| wavelength | 320 nm | 320 nm | 320 nm |
| energy (J/cm²) | 10 | 10 | 10 |
| mylar | none | none | none |
| flux | .91 | .91 | .91 |
| exposure time (minutes) | 11 | 11 | 11 |
| light configuration | VHO/Mercron | VHO/Mercron | VHO/Mercron |
| mixing speed (cpm) | 120 | 120 | 120 |
| temperature (° C.) | ˜30 | ˜30 | ˜30 |

TABLE 20

| Example 14 | | | |
|---|---|---|---|
| cell quality measurement | Day 3 | Day 4 | Day 5 |
| activation (GMP-140) decrease | 7.6 | 0 | 3.5 |
| lactate (mMol/1000 cells) | NA (15.1) | NA (3.9) | 24.93 (7.24) |
| production rate decrease % | | | |
| (production decrease %) | | | |
| glucose (mMol/1000 cells) | 5.4 | 25.6 | 37.2 |
| consumption rate decrease | | | |

TABLE 21

| | Example | | |
|---|---|---|---|
| | 15 | 15 | 15 |
| bag size | 3 L | 3 L | 3 L |
| total fluid volume (ml) | 300 | 300 | 300 |
| aspect ratio | .43 | .43 | .43 |
| blood component | platelets (1.3 × 10⁶/μl) | platelets (1.2 × 10⁶/μl) | platelets (1.4 × 10⁶/μl) |
| riboflavin (micromolar) | 50 | 50 | 50 |
| gas volume | 150 ml | 150 ml | 150 ml |
| nitric oxide | 25 ppm | 25 ppm | 25 ppm |
| nitrogen | balance | balance | balance |
| air (oxygen) | NA | NA | NA |
| photoradiation | UV | UV | UV |
| wavelength | 320 nm | 320 nm | 320 nm |
| energy (J/cm²) | 8 | 8 | 8 |
| mylar | none | none | none |
| exposure time (minutes) | 9.8 | 9.8 | 9.8 |
| flux | .91 | .91 | .91 |
| light configuration | VHO/Mercron | VHO/Mercron | VHO/Mercron |
| mixing speed (cpm) | 120 | 120 | 120 |
| temperature (° C.) | ˜30 | ˜30 | ˜30 |

TABLE 22

| Example 15 | |
|---|---|
| cell quality measurement | Day 3 |
| activation (GMP-140) decrease | 17.5 |
| HSR increase (% reversal) | 11.4 |
| lactate (mMol/1000 cells) production rate | 17.2 (32.6) |
| decrease % (production decrease %) | |
| glucose (mMol/1000 cells) consumption rate | 37.2 |
| decrease % | |
| pH rate increase % (pH unit increase) | 35.6 (.27) |
| platelet swirl increase (%) | 67 (Day 4) |

TABLE 23

| | Example | |
|---|---|---|
| | 23 | 23 |
| bag size | 3 L | 3 L |
| total fluid volume (ml) | 278 | 278 |
| aspect ratio | .4 | .4 |
| blood component | platelets (1.2 × 10⁶/μl) | platelets (1.0 × 10⁶/μl) |
| riboflavin (micromolar) | 50 | 50 |
| N-acetyl-L-cysteine | 300 μM | 300 μM |
| L-arginine | NA | NA |
| 2-deoxy-D-glucose | NA | NA |
| photoradiation | UV | UV |
| wavelength | 320 nm | 320 nm |
| energy (J/cm²) | 7 | 7 |
| mylar | none | none |
| exposure time (minutes) | 16.3 | 16.3 |
| flux | .43 | .43 |
| light configuration | T8/Mercron | T8/Mercron |
| mixing speed (cpm) | 120 | 120 |
| temperature (° C.) | ˜30 | ˜30 |

TABLE 24

| | Example | | | | | |
|---|---|---|---|---|---|---|
| | 24 | 24 | 24 | 24 | 24 | 24 |
| bag size | 3 L | 3 L | 3 L | 3 L | 3 L | 3 L |
| total fluid volume (ml) | 278 | 278 | 278 | 278 | 278 | 278 |
| aspect ratio | .4 | .4 | .4 | .4 | .4 | .4 |
| blood component | platelets | platelets | platelets | platelets | platelets | platelets |
| | (1.2 × 10⁶/μl) | (1.2 × 10⁶/μl) | (1.2 × 10⁶/μl) | (1.2 × 10⁶/μl) | (1.2 × 10⁶/μl) | (1.2 × 10⁶/μl) |
| riboflavin (micromolar) | 50 | 50 | 50 | 50 | 50 | 50 |
| L-arginine | 300 μM | 300 μM | 300 μM | 300 μM | 300 μM | 300 μM |
| 2-deoxy-D-glucose | NA | NA | NA | NA | NA | NA |
| photoradiation | UV | UV | UV | UV | UV | UV |
| wavelength | 320 nm | 320 nm | 320 nm | 320 nm | 320 nm | 320 nm |
| energy (J/cm²) | 7 | 7 | 7 | 7 | 7 | 7 |
| mylar | none | none | none | none | none | none |
| exposure time (minutes) | 15.9 | 15.9 | 15.9 | 15.9 | 15.9 | 15.9 |
| flux | .44 | .44 | .44 | .44 | .44 | .44 |
| light configuration | T8/Mercron | T8/Mercron | T8/Mercron | T8/Mercron | T8/Mercron | T8/Mercron |
| mixing speed (cpm) | 120 | 120 | 120 | 120 | 120 | 120 |
| pathogen kill (%) | NA | NA | NA | NA | NA | NA |
| temperature (° C.) | ˜30 | ˜30 | ˜30 | ˜30 | ˜30 | ˜30 |

TABLE 25

| | Example | | | |
|---|---|---|---|---|
| | 25 | 25 | 25 | 25 |
| bag size | 3 L | 3 L | 3 L | 3 L |
| total fluid volume (ml) | 278 | 278 | 278 | 278 |
| aspect ratio | .4 | .4 | .4 | .4 |
| blood component | platelets (1.2 × 10⁶/μl) | platelets (1.3 × 10⁶/μl) | platelets (1.2 × 10⁶/μl) | platelets (1.3 × 10⁶/μl) |
| riboflavin (micromolar) | 50 | 50 | 50 | 50 |
| N-acetyl-L-cysteine | 300 μM | 300 μM | 300 μM | 300 μM |
| L-arginine | NA | NA | NA | NA |
| 2-deoxy-D-glucose | NA | NA | NA | NA |
| photoradiation | UV | UV | UV | UV |
| wavelength | 320 nm | 320 nm | 320 nm | 320 nm |
| energy (J/cm²) | 7 | 7 | 7 | 7 |
| mylar | none | none | none | none |
| exposure time (minutes) | 15.9 | 15.9 | 15.9 | 15.9 |
| flux | .44 | .44 | .44 | .44 |
| light configuration | T8/Mercron | T8/Mercron | T8/Mercron | T8/Mercron |
| mixing speed (cpm) | 120 | 120 | 120 | 120 |
| temperature (° C.) | ˜30 | ˜30 | ˜30 | ˜30 |

TABLE 26

| | Example | | | | | |
|---|---|---|---|---|---|---|
| | 26 | 26 | 26 | 26 | 26 | 26 |
| bag size | 3 L | 3 L | 3 L | 3 L | 3 L | 3 L |
| total fluid volume (ml) | 278 | 278 | 278 | 278 | 278 | 278 |
| aspect ratio | .4 | .4 | .4 | .4 | .4 | .4 |
| blood component | platelets | platelets | platelets | platelets | platelets | platelets |
| | (1.1 × 10⁶/μl) | (1.2 × 10⁶/μl) | (1.2 × 10⁶/μl) | (1.3 × 10⁶/μl) | (1.1 × 10⁶/μl) | (1.2 × 10⁶/μl) |
| riboflavin (micromolar) | 50 | 50 | 50 | 50 | 50 | 50 |
| N-acetyl-L-cysteine | NA | NA | NA | NA | NA | NA |
| L-arginine | 300 μM | 300 μM | 300 μM | 300 μM | 300 μM | 300 μM |
| 2-deoxy-D-glucose | 10 mM | 10 mM | 10 mM | 10 mM | 10 mM | 10 mM |
| photoradiation | UV | UV | UV | UV | UV | UV |
| wavelength | 320 nm | 320 nm | 320 nm | 320 nm | 320 nm | 320 nm |
| energy (J/cm²) | 7 | 7 | 7 | 7 | 7 | 7 |
| mylar | none | none | none | none | none | none |
| exposure time (minutes) | 15.9 | 15.9 | 15.9 | 15.9 | 15.9 | 15.9 |
| flux | .44 | .44 | .44 | .44 | .44 | .44 |
| light configuration | T8/Mercron | T8/Mercron | T8/Mercron | T8/Mercron | T8/Mercron | T8/Mercron |
| mixing speed (cpm) | 120 | 120 | 120 | 120 | 120 | 120 |
| temperature (° C.) | ˜30 | ˜30 | ˜30 | ˜30 | ˜30 | ˜30 |

TABLE 27

| | Example |
|---|---|
| | 27 |
| bag size | 3 L |
| total fluid volume (ml) | 278 |
| aspect ratio | .4 |
| blood component | platelets (1.3 × 10⁶/μl) |
| riboflavin (micromolar) | 50 |
| N-acetyl-L-cysteine | NA |
| L-arginine | 300 μM |
| 2-deoxy-D-glucose | 10 mM |
| photoradiation | UV |
| wavelength | 320 nm |
| energy (J/cm²) | 7 |
| mylar | none |
| exposure time (minutes) | 16.3 |
| flux | .43 |
| light configuration | T8/Mercron |
| mixing speed (cpm) | 120 |
| temperature (° C.) | ˜30 |

TABLE 28

| | Example | | | | | |
|---|---|---|---|---|---|---|
| | 28 | 28 | 28 | 28 | 28 | 28 |
| bag size | 3 L | 3 L | 3 L | 3 L | 3 L | 3 L |
| total fluid volume (ml) | 278 | 278 | 278 | 278 | 278 | 278 |
| aspect ratio | .4 | .4 | .4 | .4 | .4 | .4 |
| blood component | platelets | platelets | platelets | platelets | platelets | platelets |
| | (1.0 × 10⁶/μl) | (1.2 × 10⁶/μl) | (1.2 × 10⁶/μl) | (1.2 × 10⁶/μl) | (1.2 × 10⁶/μl) | (1.2 × 10⁶/μl) |
| riboflavin (micromolar) | 50 | 50 | 50 | 50 | 50 | 50 |
| N-acetyl-L-cysteine | 300 μM | 300 μM | 300 μM | 300 μM | 300 μM | 300 μM |
| L-arginine | NA | NA | NA | NA | NA | NA |
| 2-deoxy-D-glucose | 10 mM | 10 mM | 10 mM | 10 mM | 10 mM | 10 mM |
| photoradiation | UV | UV | UV | UV | UV | UV |
| wavelength | 320 nm | 320 nm | 320 nm | 320 nm | 320 nm | 320 nm |
| energy (J/cm²) | 7 | 7 | 7 | 7 | 7 | 7 |
| mylar | none | none | none | none | none | none |
| exposure time (minutes) | 15.9 | 15.9 | 15.9 | 15.9 | 15.9 | 15.9 |
| flux | .44 | .44 | .44 | .44 | .43 | .43 |
| light configuration | T8/Mercron | T8/Mercron | T8/Mercron | T8/Mercron | T8/Mercron | T8/Mercron |
| mixing speed (cpm) | 120 | 120 | 120 | 120 | 120 | 120 |
| temperature (° C.) | ˜30 | ˜30 | ˜30 | ˜30 | ˜30 | ˜30 |

TABLE 29

| | Example | | | | | |
|---|---|---|---|---|---|---|
| | 29 | 29 | 29 | 29 | 29 | 29 |
| bag size | 3 L | 3 L | 3 L | 3 L | 3 L | 3 L |
| total fluid volume (ml) | 278 | 278 | 278 | 278 | 278 | 278 |
| aspect ratio | .4 | .4 | .4 | .4 | .4 | .4 |
| blood component | platelets | platelets | platelets | platelets | platelets | platelets |
| | (1.1 × 10⁶/μl) | (1.2 × 10⁶/μl) | (1.2 × 10⁶/μl) | (1.1 × 10⁶/μl) | (1.1 × 10⁶/μl) | (1.2 × 10⁶/μl) |
| riboflavin (micromolar) | 50 | 50 | 50 | 50 | 50 | 50 |
| N-acetyl-L-cysteine | 300 μM | 300 μM | 300 μM | 300 μM | 300 μM | 300 μM |
| L-arginine | 300 μM | 300 μM | 300 μM | 300 μM | 300 μM | 300 μM |
| 2-deoxy-D-glucose | 10 mM | 10 mM | 10 mM | 10 mM | 10 mM | 10 mM |
| photoradiation | UV | UV | UV | UV | UV | UV |
| wavelength | 320 nm | 320 nm | 320 nm | 320 nm | 320 nm | 320 nm |
| energy (J/cm²) | 7 | 7 | 7 | 7 | 7 | 7 |
| mylar | none | none | none | none | none | none |
| exposure time (minutes) | 15.9 | 15.9 | 15.9 | 15.9 | 15.9 | 15.9 |
| flux | .44 | .44 | .44 | .44 | .44 | .44 |
| light configuration | T8/Mercron | T8/Mercron | T8/Mercron | T8/Mercron | T8/Mercron | T8/Mercron |
| mixing speed (cpm) | 120 | 120 | 120 | 120 | 120 | 120 |
| temperature (° C.) | ˜30 | ˜30 | ˜30 | ˜30 | ˜30 | ˜30 |

TABLE 30

| | Example | | |
|---|---|---|---|
| | 30 | 30 | 30 |
| bag size | 3 L | 3 L | 3 L |
| total fluid volume (ml) | 278 | 278 | 278 |
| aspect ratio | .4 | .4 | .4 |
| blood component | plasma (85%) | plasma (85%) | plasma (85%) |
| riboflavin (micromolar) | 50 | 50 | 50 |
| N-acetyl-L-cysteine | NA | NA | NA |
| L-arginine | 300 μM | 300 μM | 300 μM |
| 2-deoxy-D-glucose | NA | NA | NA |
| photoradiation | UV | UV | UV |
| wavelength | 320 nm | 320 nm | 320 nm |
| energy (J/cm²) | 5, 7, 9 | 5, 7, 9 | 5, 7, 9 |
| mylar | none | none | none |
| exposure time (minutes) | 11.4, 15.9, 20.4 | 11.4, 15.9, 20.4 | 11.4, 15.9, 20.4 |
| flux | .44 | .44 | .44 |
| light configuration | T8/Mercron | T8/Mercron | T8/Mercron |
| mixing speed (cpm) | 120 | 120 | 120 |
| pathogen | BVDV | BVDV | BVDV |
| pathogen titer (log, T0) | 6.24 | 5.92 | 6.24 |
| pathogen titer (log, T1) | 3.83 | 3.29 | 3.57 |
| pathogen kill (log, T1 − T0) | −2.41 | −2.63 | −2.67 |
| pathogen kill at T1 (%) | 99.6 | 99.77 | 99.79 |
| pathogen titer (log, T2) | 2.9 | 2.6 | 2.73 |
| pathogen kill (log, T2 − T0) | −3.34 | −3.32 | −3.51 |
| pathogen kill at T2 (%) | 99.95 | 99.95 | 99.97 |
| pathogen titer (log, T3) | 2.6 | 2.0 | 2.4 |
| pathogen kill (log, T3 − T0) | −3.64 | −3.92 | −3.84 |
| pathogen kill at T3 (%) | 99.98 | 99.99 | 99.99 |
| temperature (° C.) | ˜30 | ˜30 | ˜30 |

TABLE 31

| | Example | | |
|---|---|---|---|
| | 31 | 31 | 31 |
| bag size | 3 L | 3 L | 3 L |
| total fluid volume (ml) | 278 | 278 | 278 |
| aspect ratio | .4 | .4 | .4 |
| blood component | plasma (85%) | plasma (85%) | plasma (85%) |
| riboflavin (micromolar) | 50 | 50 | 50 |
| N-acetyl-L-cysteine | 300 μM | 300 μM | 300 μM |
| L-arginine | NA | NA | NA |
| 2-deoxy-D-glucose | NA | NA | NA |
| photoradiation | UV | UV | UV |
| wavelength | 320 nm | 320 nm | 320 nm |
| energy (J/cm²) | 5, 7, 9 | 5, 7, 9 | 5, 7, 9 |
| mylar | none | none | none |
| exposure time (minutes) | 11.4, 15.9, 20.4 | 11.4, 15.9, 20.4 | 11.4, 15.9, 20.4 |
| flux | .44 | .44 | .44 |
| light configuration | T8/Mercron | T8/Mercron | T8/Mercron |
| mixing speed (cpm) | 120 | 120 | 120 |
| pathogen | BVDV | BVDV | BVDV |
| pathogen titer (log, T0) | 6.5 | 6.38 | 6.5 |
| pathogen titer (log, T1) | 3.63 | 4.08 | 3.79 |
| pathogen kill (log, T1 − T0) | −2.87 | −2.3 | −2.71 |
| pathogen kill at T1 (%) | 99.87 | 99.5 | 99.8 |
| pathogen titer (log, T2) | 2.9 | 3.35 | 2.9 |
| pathogen kill (log, T2 − T0) | −3.6 | −3.0 | −3.6 |
| pathogen kill at T2 (%) | 99.98 | 99.91 | 99.98 |
| pathogen titer (log, T3) | 2.6 | 2.6 | 2.6 |
| pathogen kill (log, T3 − T0) | −3.9 | −3.78 | −3.9 |
| pathogen kill at T3 (%) | 99.98 | 99.98 | 99.99 |
| temperature (° C.) | ˜30 | ˜30 | ˜30 |

TABLE 32

| | Example |
|---|---|
| | 32 |
| bag size | 3 L |
| total fluid volume (ml) | 278 |
| aspect ratio | .4 |
| blood component | plasma (85%) |
| riboflavin (micromolar) | 50 |
| N-acetyl-L-cysteine | 300 μM |
| L-arginine | NA |
| 2-deoxy-D-glucose | 10 mM |
| photoradiation | UV |
| wavelength | 320 nm |
| energy (J/cm²) | 5, 7, 9 |
| mylar | none |
| exposure time (minutes) | 11.4, 15.9, 20.4 |
| flux | .44 |
| light configuration | T8/Mercron |
| mixing speed (cpm) | 120 |
| pathogen | BVDV |
| pathogen titer (log, T0) | 6.5 |
| pathogen titer (log, T1) | 3.79 |
| pathogen kill (log, T1 − T0) | −2.71 |
| pathogen kill at T1 (%) | 99.8 |
| pathogen titer (log, T2) | 2.9 |
| pathogen kill (log, T2 − T0) | −3.6 |
| pathogen kill at T2 (%) | 99.97 |
| pathogen titer (log, T3) | 2.6 |
| pathogen kill (log, T3 − T0) | −3.9 |
| pathogen kill at T3 (%) | 99.99 |
| temperature (° C.) | ˜30 |

TABLE 33

| | | 300 μM | 300 μM L-Arg + 2- | 300 μM NAC + | 300 μM L-Arg + 300 μM | |
|---|---|---|---|---|---|---|
| Examples 23-31 | 300 μM NAC | L-Arg | DOG | 2-DOG | NAC + 10 mM 2-DOG | 10 mM 2-DOG |
| activation (GMP-140) | 32.3 | 40.8 | 45.2 | NA | 45.2 | 32.3 |
| decrease at Day 1 | | | | | | |
| HSR increase (% reversal) | NA | 32.7 | NA | 44.4 | NA | NA |
| at Day 5 | | | | | | |
| lactate (mMol/1000 cells) | 18.8 | 6.97 | 65.13 | 60.27 | 57.04 | 57.53 |
| production rate decrease % | (NA) | (4.07) | (46.23) | (12.63) | (34.47) | (40.16) |
| at Day 5 (production | | | | | | |
| decrease %) | | | | | | |
| glucose (mMol/1000 cells) | 25.6 | 21/6 | 75.4 | 70.2 | 61.6 | 79.2 |
| consumption rate decrease | | | | | | |
| % at Day 5 | | | | | | |
| pH rate increase % (pH | 0.03 | 0.03 | 0.69 | NA | 0.55 | 0.61 |
| unit increase) | | | | | | |
| platelet swirl increase (%) | 10.4 | NA | 32.1 | 41.5 | 88.7 | 41.5 |
| ESC increase (%) | NA | NA | 2.2 | NA | 24.4 | NA |

This invention has been illustrated using particular components, reagents and method steps. Equivalents known to the art can be substituted for any of these and are included within the scope of the following claims.

We claim:

1. A method for treating a fluid comprising a blood component to inactivate pathogens which may be present therein, comprising the steps of:
(a) adding an inactivation-effective, substantially non-toxic amount of a photosensitizer to said fluid;
(b) adding nitric oxide to said fluid in an amount sufficient to increase dissolved nitric oxide content of said fluid and in an amount sufficient to improve a vital quality of said blood component; and
(c) exposing said fluid to photoradiation of sufficient energy to activate said photosensitizer, for a sufficient time to substantially inactivate said pathogens.

2. The method of claim 1 wherein said increased dissolved nitric oxide content of said fluid is in an amount sufficient to also increase pathogen inactivation.

3. The method of claim 1 wherein the step of adding nitric oxide is performed by adding nitric oxide gas, adding one or more nitric oxide generators, or both.

4. The method of claim 1 wherein the step of adding nitric oxide is performed by adding nitric oxide gas, adding L-arginine, adding N-acetyl-cysteine, adding dibutyryl guanosine monophosphate, and/or adding dibutyryl adenosine monophosphate.

5. The method of claim 1 wherein the step of adding nitric oxide comprises adding nitric oxide gas.

6. The method of claim 1 wherein the step of adding nitric oxide comprises adding L-arginine.

7. The method of claim 6 comprising adding between about 100 micromolar and about 500 micromolar L-arginine.

8. The method of claim 6 comprising adding about 100 micromolar L-arginine.

9. The method of claim 1 wherein adding nitric oxide comprises adding N-acetyl-cysteine.

10. The method of claim 9 wherein said N-acetyl-cysteine is N-acetyl-L-cysteine.

11. The method of claim 9 comprising adding between about 100 micromolar and about 500 micromolar N-acetyl-cysteine.

12. The method of claim 9 comprising adding about 100 micromolar N-acetyl-cysteine.

13. The method of claim 1 further comprising the step of increasing the dissolved oxygen content of said fluid to an amount sufficient to enhance reaction of the photosensitizer in which singlet oxygen and reactive oxygen species (ROS) are formed.

14. The method of claim 1 further comprising the step of adding a quencher to said fluid.

15. The method of claim 1 further comprising the step of adding a glycolysis inhibitor to said fluid.

16. The method of claim 15 wherein said glycolysis inhibitor is 2-deoxy-D-glucose.

17. The method of claim 16 wherein said 2-deoxy-D-glucose is present at a concentration between about 1 millimolar and about 10 millimolar.

18. The method of claim 1 wherein said fluid comprises a blood component selected from the group consisting of plasma, platelets, red blood cells, white blood cells, and plasma proteins.

19. The method of claim 18 wherein said fluid comprises platelets.

20. The method of claim 19 wherein said fluid comprises platelets in a solution comprising plasma and storage solution.

21. The method of claim 1 performed in a blood product collection bag.

22. The method of claim 1 wherein said photosensitizer is selected from the group consisting of endogenous isoalloxazines and isoalloxazine derivative photosensitizers.

23. The method of claim 1 wherein said photosensitizer is riboflavin.

24. The method of claim 1 wherein the concentration of said photosensitizer in said fluid is about 50 micromolar.

25. The method of claim 5 comprising adding an amount of nitric oxide gas equivalent to between about 25 ml and about 1200 ml at about 1 atmosphere of pressure.

26. The method of claim 25 wherein the amount of nitric oxide gas is about 150 ml.

27. The method of claim 25 wherein the concentration of nitric oxide in said gas is between about 10 ppm and about 1000 ppm.

28. The method of claim 25 wherein the concentration of nitric oxide in said gas is between about 25 ppm and about 50 ppm.

29. The method of claim 5 wherein said nitric oxide gas is in a balance of about 20% oxygen and about 80% nitrogen.

30. The method of claim 1 wherein said vital quality of said blood component is selected from the group consisting of activation of said blood component, hypotonic shock response, lactate production, glucose consumption, pH, platelet swirl, and platelet aggregation.

31. The method of claim 1 wherein said pathogens are selected from the group consisting of extracellular and intracellular viruses, bacteria, bacteriophages, fungi, blood-transmitted parasites, and protozoa, and mixtures of any two or more of the foregoing.

32. The method of claim 31 wherein said viruses are selected from the group consisting of human immunodeficiency virus (HIV), hepatitis A, B and C viruses, sindbis virus, cytomegalovirus, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, and Epstein-Barr virus, bovine viral diarrhea virus, pseudorabies, West Nile virus, and mixtures of any two or more of the foregoing.

33. The method of claim 31 wherein said bacteriophages are selected from the group consisting of ΦX174, Φ6, λ, R17, T₄, and T₂, and mixtures of any two or more of the foregoing.

34. The method of claim 31 wherein said bacteria are selected from the group consisting of P. aeruginosa, S. aureus, S. epidermidis, E. coli, K. pneumoniae, E. faecalis, B. subtilis, S. pneumoniae, S. pyrogenes, S. viridans, B. cereus, E. aerogenes, propionabacter, C. perfringes, E. cloacae, P. mirabilis, S. cholerasuis, S. liquifaciens, S. mitis, Y. entercolitica, P. fluorescens, S. enteritidis, C. freundii, and S. marcescens, and mixtures of any two or more of the foregoing.

35. The method of claim 31 wherein said protozoa is P. falciparum.

36. A blood product decontaminated by the method of claim 1.

37. A biological composition comprising:
(a) a fluid;
(b) an inactivation-effective, substantially non-toxic amount of an endogenous photosensitizer or endogenously-based derivative photosensitizer; and
(c) dissolved nitric oxide in an amount greater than would be present in said fluid under an air atmosphere at ambient conditions without mixing.

38. The composition of claim 37 also comprising dissolved oxygen in an amount greater than would be present under an air atmosphere at ambient conditions without mixing.

39. The composition of claim 37 also comprising a quencher.

40. The composition of claim 37 also comprising a glycolysis inhibitor.

41. The composition of claim 37 wherein said fluid comprises one or more blood components selected from the group consisting of plasma, red blood cells, white blood cells, platelets and plasma proteins.

42. The composition of claim 37 wherein said fluid comprises platelets.

43. The composition of claim 37 wherein said fluid comprises platelets and storage solution.

44. The composition of claim 37 also comprising pathogens.

45. The composition of claim 44 wherein said pathogens are selected from the group consisting of extracellular and intracellular viruses, bacteria, bacteriophages, fungi, blood-transmitted parasites, and protozoa, and mixtures of any two or more of the foregoing.

46. The composition of claim 44 in which said pathogens have been substantially inactivated.

47. A translucent or transparent blood component bag wherein the contents of said blood component bag comprise the composition of claim 37.

48. The blood component bag of claim 47 wherein the contents of which also comprise air.

49. The blood component bag of claim 47 wherein the contents of which also comprise more oxygen than is present in air.

50. The blood component bag of claim 47 wherein the contents of which also comprise more nitric oxide than is present in air.

51. The blood component bag of claim 47 wherein the contents of which also comprise more oxygen and more nitric oxide than is present in air.

52. A decontamination system for a fluid comprising:
(a) a leak-proof transparent or translucent container for the fluid;
(b) a photosensitizer source for providing photosensitizer to said container, said photosensitizer source being connectible to an inlet of said container;
(c) a nitric oxide source connectible to an inlet of said container for providing nitric oxide to said container; and
(d) a photoirradiator for irradiating said container.

53. The decontamination system of claim 52 also comprising an oxygen source connectible to an inlet of said container for providing oxygen to said container.

54. The decontamination system of claim 52 also comprising a quencher source for providing quencher to said container, said quencher source being connectible to an inlet of said container.

55. The decontamination system of claim 52 also comprising a glycolysis inhibitor source for providing glycolysis inhibitor to said container, said glycolysis inhibitor source being connectible to an inlet of said container.

56. The decontamination system of claim 52 also comprising an agitator for agitating said container.

57. The decontamination system of claim 52 wherein said container is a blood product or blood collection bag.

58. The decontamination system of claim 57 wherein said blood product bag or blood collection bag contains said fluid wherein said fluid comprises a blood component selected from the group consisting of plasma, platelets, red blood cells, white blood cells, and plasma proteins.

59. The decontamination system of claim 57 wherein said nitric oxide source contains a composition selected from the group consisting of nitric oxide gas, L-arginine, and N-acetyl-cysteine.

60. The decontamination system of claim 52 wherein said blood component is platelets.

61. A method for making a decontamination system comprising the steps of:
(a) providing a set of components comprising:
(i) a leak-proof transparent or translucent container for a fluid wherein said container comprises one or more inlets;
(ii) a photosensitizer source for providing photosensitizer to said container, said photosensitizer source comprising an outlet connectible to one of said inlets of said container;
(iii) a nitric oxide source for providing nitric oxide to said container, said nitric oxide source comprising an outlet connectible to one of said inlets of said container; and
(iv) a photoirradiator for irradiating said container;
(b) connecting the outlet of said photosensitizer source to an inlet of said container;
(c) connecting the outlet of said nitric oxide source to an inlet of said container; and
(d) positioning said photoirradiator in radiating proximity to said container.

62. The method of claim 61 wherein said set of components also comprises an oxygen source having an outlet for providing oxygen to said container, said oxygen source being connectible to an inlet of said container, and said method comprises connecting the outlet of said oxygen source to an inlet of said container.

63. A method of increasing the storage life of platelets, said method comprising the steps of:
(a) placing said platelets in solution in a container larger than the volume of the solution;
(b) adding nitric oxide to said solution to increase dissolved nitric oxide content of said solution in an amount sufficient to improve storage life of said platelets;
(c) adding a photoactivator to said solution and irradiating said solution to activate said photoactivator; and
(d) storing said platelets for a period greater than five days without destroying the usefulness of said platelets.

64. The method of claim 63 also comprising dissolving an amount of oxygen in said solution greater than that would be dissolved in said solution under an air atmosphere at ambient conditions without agitation.

65. The method of claim 63 further comprising removing substantially all gas phase from said container after step (c).

66. The method of step of claim 65 further comprising adding nitric oxide to said solution after step (c) to increase dissolved nitric oxide content of said solution in an amount sufficient to further increase storage life of said blood component.

67. The method step of claim 63 further comprising adding a glycolysis inhibitor to said solution in an amount sufficient to further increase storage life of said blood component.

68. The method of claim 63 further comprising adding a quencher to said solution.

69. A method for treating platelets to inactivate pathogens which may be present therein, comprising the steps of:
(a) adding 7,8-dimethyl-10-ribityl isoalloxazine to a fluid comprising said platelets in storage solution at a ratio of about 27:73 platelets (in plasma):storage solution, whereby the 7,8-dimethyl-10-ribityl isoalloxazine concentration of said fluid is between about 1 and about 200 micromolar;
(b) adding nitric oxide gas to the atmosphere in contact with said fluid to increase dissolved nitric oxide content of said fluid in an amount sufficient to increase a vital quality of said platelets, wherein the concentration of said nitric oxide gas is between about 10 ppm and about 500 ppm; and
(c) exposing said fluid to photoradiation at an energy between about 5 and about 12 J/cm² to activate the photosensitizer, for at least about five to about twenty minutes, to substantially inactivate said pathogens.

70. The method of claim 69 further comprising increasing the dissolved oxygen content of said fluid to about five times the oxygen content said fluid would have under an air atmosphere, by mixing air into said fluid, or by exposing said fluid to an atmosphere of substantially pure oxygen, before step (c).

71. The method of claim 69 further comprising adding quencher to said fluid.

72. The method of claim 69 further comprising adding glycolysis inhibitor to said fluid.

73. The method of claim 69 further comprising adding L-arginine to said fluid.

74. The method of claim 69 further comprising adding N-acetyl-cysteine to said fluid.

75. A method for treating platelets to inactivate pathogens which may be present therein, comprising the steps of:
(a) adding 7,8-dimethyl-10-ribityl isoalloxazine to a fluid comprising said platelets in storage solution at a ratio of about 27:73 platelets (in plasma):storage solution, whereby the 7,8-dimethyl-10-ribityl isoalloxazine concentration of said fluid is between about 1 and about 200 micromolar;
(b) adding L-arginine to said fluid in an amount sufficient to increase a vital quality of said platelets, wherein the concentration of said L-arginine is between about 25 micromolar and about 500 micromolar; and
(c) exposing said fluid to photoradiation at an energy between about 5 and about 12 J/cm² to activate the photosensitizer, for at least about five to about twenty minutes, to substantially inactivate said pathogens.

76. The method of claim 75 further comprising the step of increasing the dissolved oxygen content of said fluid to about five times the oxygen content the fluid would have under an air atmosphere, by mixing air into said fluid, or by exposing said fluid to an atmosphere of substantially pure oxygen, before step (c).

77. The method of claim 75 further comprising the step of adding quencher to said fluid.

78. The method of claim 75 further comprising the step of adding glycolysis inhibitor to said fluid.

79. The method of claim 75 further comprising the step of adding nitric oxide gas to said fluid.

80. The method of claim 75 further comprising the step of adding N-acetyl-cysteine to said fluid.

81. A method for treating platelets to inactivate pathogens which may be present therein, comprising the steps of:
(a) adding 7,8-dimethyl-10-ribityl isoalloxazine to a fluid comprising said platelets in storage solution at a ratio of about 27:73 platelets (in plasma):storage solution, whereby the 7,8-dimethyl-10-ribityl isoalloxazine concentration of said fluid is between about 1 and about 200 micromolar;
(b) adding N-acetyl-cysteine to said fluid in an amount sufficient to increase a vital quality of said platelets, wherein the concentration of said N-acetyl-cysteine is between about 25 micromolar and about 500 micromolar; and
(c) exposing said fluid to photoradiation at an energy between about 5 and about 12 J/cm² to activate the photosensitizer, for at least about five to about twenty minutes, to substantially inactivate said pathogens.

82. The method of claim 81 further comprising the step of increasing the dissolved oxygen content of said fluid to about five times the oxygen content the fluid would have under an air atmosphere, by mixing air into said fluid, or by exposing said fluid to an atmosphere of substantially pure oxygen, before step (c).

83. The method of claim 81 further comprising the step of adding quencher to said fluid.

84. The method of claim 81 further comprising the step of adding glycolysis inhibitor to said fluid.

85. The method of claim 81 further comprising the step of adding L-arginine to said fluid.

86. The method of claim 81 further comprising the step of adding nitric oxide gas to said fluid.

87. The method of claim 1 wherein the step of adding nitric oxide comprises adding a nitric oxide donor.

88. The method of claim 1 wherein the step of adding nitric oxide comprises adding a composition selected from the group consisting of: DEA-NO, DETA-NO, DETA-NONOate, PAPA-NO, sodium nitroprusside, and nitroglycerine.

89. The method of claim 1 wherein the step of adding nitric oxide is performed before, after, or both before and after the step of exposing said fluid to photoradiation.

90. The method of claim 14 wherein the step of adding a quencher is performed before, after, or both before and after the step of exposing said fluid to photoradiation.

91. The method of claim 15 wherein the step of adding a glycolysis inhibitor is performed before, after, or both before and after the step of exposing said fluid to photoradiation.

92. A blood product decontaminated by the method of claim 1 wherein said blood product is suitable for administration for a patient for at least about five days.

93. A blood product decontaminated by the method of claim 1 wherein said blood product is suitable for administration for a patient for more than five days.

94. The bag of claim 47 wherein said bag comprises a nitric oxide generator.

95. The bag of claim 47 wherein said bag comprises a coating wherein said coating comprises a nitric oxide generator.

96. A method of increasing the storage life of a blood component, said method comprising the steps of:
(a) placing said blood component in solution in a container larger than the volume of the solution;
(b) adding nitric oxide to said solution to increase dissolved nitric oxide content of said solution in an amount sufficient to improve storage life of said blood component;
(c) adding a photoactivator to said solution and irradiating said solution to activate said photoactivator; and
(d) storing said blood component for a period greater than five days without destroying the usefulness of said blood component.
